(19) United States
(12) Patent Application Publication
(10) Pub. No.: US 2026/0234690 A1
(43) Pub. Date: Aug. 13, 2026

(54) ENZYME LINKED IMMUNOASSAY WITH DIFFUSION RATE LIMITING MEDIUM

(71) Applicant: ELLEON BIOTECH LIMITED, Hong Kong (CN)

(72) Inventors: Chi Wai TSANG, Hong Kong (CN); Nga Chi YIP, Hong Kong (CN)

(21) Appl. No.: 19/472,706

(22) PCT Filed: Sep. 19, 2024
(86) PCT No.: PCT/IB2024/059086

§ 371 Date: Oct. 5, 2025

(30) Foreign Application Priority Data
Sep. 22, 2023 (HK) 32023079605.6

(51) Int. Cl. C12Q 1/06; C12Q 1/04; C12Q 1/24

(52) U.S. Cl. CPC C12Q 1/06; C12Q 1/045; C12Q 1/24

(57) ABSTRACT

An aspect of the present invention provides a method for detecting a target organism in a sample, the method comprising: a. optionally immobilizing the target organism to a solid phase; b. incubating the target organism with one or more reporter enzyme detection probes to form one or more target organism-enzyme detection probe complexes; c. removing any unbound reporter enzyme detection probes; d. contacting the one or more target organism-enzyme detection probe complexes with a diffusion rate limiting medium containing one or more substrates and incubating the one or more target organism-enzyme detection probe complexes with the one or more substrates in the diffusion rate limiting medium to generate one or more detectable products in the diffusion rate limiting medium; e. detecting the one or more detectable products by detecting visible spots.

### TECHNICAL FIELD

The invention relates to an enzyme linked immunoassay with diffusion rate limiting medium. Specifically, the invention relates to a method for detecting a target organism in a sample.

### BACKGROUND ART

Diverse investigative procedures and assays are employed in fields of medicine, molecular biology, and so forth. For example, assays are widely used in immunology for determining rate of activation of cells in response to vaccines, infections, allergens, etc. Assays involving antibodies as key components are typically referred to as immunoassays, which are the preferred analytical method for the repetitive quantitative analysis of biomolecules, such as polypeptides molecules, of biomedical importance. Examples of immunoassays include, but are not limited to, Enzyme-Linked Immunosorbent Assay (ELISA), Enzyme-Linked ImmunoSpot (ELISpot) assay, FluoroSpot assay.

ELISpot is used for quantification of cells that have a targeted secretion marker. It is limited to secretion markers only requiring capture of the secretions by a specific probe. The capture antibody is bound to the membrane on the platform/plate and thus binds the secretion to the membrane also. Cells are then washed away, and the platform treated with a probe and substrate which in the presence of the secretion marker will form a coloured spot on the membrane at the location of binding. The following three main problems can be identified with ELISpot. First the membrane coating on the platform significantly increases the cost per assay over using standard platforms. Secondly, the requirement to wash away the cells means that surface markers cannot be targeted. Thirdly, this means target cells are removed and cannot be harvested for further use.

ELISA is a quantitative and qualitative immunoassay commonly used for detecting biomolecules such as antigens, antibodies, cytokines etc. in an overall population of cells. Cell-based ELISA can also detect cell surface markers. However, during ELISA assays, cells are either fixed (a process in which the cell is perforated and therefor dead) or washed away. Quantitative ELISA is limited to relative quantification by pure reference colour concentration calibration comparison. Therefore, it cannot distinguish the exact number of cells presenting the cell surface marker or secreting marker of interest. Being a population study sufficient detection limit must be reached to give a detectable result. Small positive results within a large sample my remain undetectable thus giving false negative results.

Flowcytometry is used for quantification of cells within a large population which have specific cell surface markers. More recently flowcytometry has been used for cell secretion markers also but requires more steps to tag cell surface markers which can then trap the secretion marker. The equipment for the assay is very expensive and whilst cells can be sorted after analysis the process can cause a high percentage of cell loss and does not allow isolation of an individual cell without further isolation technique.

Thus, there is a significant unmet need for an inexpensive single assay which can be used for analysis of cell surface markers, cell secretion markers, or combinations of both, which can isolate cells down to a single cell, and maintain cell viability. Requiring little to no expensive equipment and can be performed on any platform form for example petri-dish, 96 well plates, or even clear vials.

### SUMMARY OF INVENTION

An aspect of the present invention provides a method for detecting a target organism in a sample, the method comprising
a. optionally immobilizing the target organism to a solid phase;
b. incubating the target organism with one or more reporter enzyme detection probes to form one or more target organism-enzyme detection probe complexes;
c. removing any unbound reporter enzyme detection probes;
d. contacting the one or more target organism-enzyme detection probe complexes with a diffusion rate limiting medium containing one or more substrates and incubating the one or more target organism-enzyme detection probe complexes with the one or more substrates in the diffusion rate limiting medium to generate one or more detectable products; and
e. detecting the one or more detectable products by detecting visible spots;
wherein the diffusion rate limiting medium is any suitable medium that decreases diffusion rate of the one or more detectable products and thereby allowing formation of the visible spots.

Another aspect of the present invention provides a method of quantifying the amount of a target organism in a sample, the method comprising
a. detecting the target organism according to the method of the invention; and
b. quantifying the amount of the target organism in the sample based on the number of visible spots.

### BRIEF DESCRIPTION OF DRAWINGS

FIG. 1 shows the general concept of the method according to the present invention for detecting a target organism in a sample.

FIG. 2 shows [A] ELISpot for detecting a target substance that is secreted by the target organism; [B] method according to the present invention for detecting a target substance that is secreted by the target organism via platform selective capturing; [C] method according to the present invention for detecting a target substance that is secreted by the target organism via surface marker tagging.

FIG. 3 shows a method according to the present invention where two or optionally three markers can be used to specifically identify cells with targeted attributes eliciting a single positive or negative response for example the presence or not of a colourimetric change.

FIG. 4 shows a method according to the present invention where three markers are used to specifically identify cells with combinations of multiple attributes eliciting multiple possible responses. The use of different colourimetric, fluorescent, or other substrate to product reactions can give rise to combinations of responses and just some are depicted here, [A] shows response from all three markers; [B] shows response from two out of three possible markers; [C] shows response from just one of three possible markers. Other combinations are possible this is just meant as possible examples.

FIG. 5 shows two further embodiments of the present invention as described in FIG. 1 depicting alternative application of the diffusion rate limiting medium with substrate. [A] shows an example application of the diffusion rate limiting medium on top of the sample which have been treated with a reporter enzyme detection probe. [B] shows an example application where a platform can be prepared with the diffusion rate limiting medium and have the sample pretreated with the reporter enzyme detection probe applied on to the surface.

FIG. 6 shows diagrammatic depiction of number of countable visible spots (dots) (y-axis) vs diffusion rate of substrate and detectable product as controlled by the variable listed herein (x-axis).

FIG. 7 shows a representative picture of methylene blue diffusion through different agar gel compositions; 0.25%, 0.5%, 0.75%, 1% and 1.5% (W/V) agar in water. Each gel composition was prepared 3 times (apart from 0.5%, n=2) and measurements of methylene blue diffusion (distance diffused in pixels shown with white arrows) for each gel was recorded 5 times. Summary of methylene blue diffusion distance after 1 hour shown in [FIG. 8].

FIG. 8 shows methylene blue diffusion vs agar composition plot. (n=3).

FIG. 9 shows photos taken with a macro ccd camera of rows i) ELISpot wells, ii) ELISA wells and iii) diagrammatic depiction of the different layers in the experiment with columns A) and B) (reading from bottom up) wells were coated with (1) primary Rabbit anti-human CD27 antibodies, (2) CD27+ cells, (3) Rabbit anti-human CD27 antibodies labelling and (4) secondary HRP conjugated goat anti-rabbit Fc region antibodies. Column C) reading from bottom up, of a well coated with (1) primary Rabbit monoclonal antibody (mAb) anti-CD27 antibodies and (2) CD27+ cells, and no further labelling. Pictures Ai) and Aii) were developed with 50:50 (TMB: 2×RPMI), Bi) and Ci) were developed with 1:1:2 (4×RPMI: 1.2% agar: TMB), and Bii) and Cii) were developed with 1:1:2 (4×RPMI: 1.2% agarose: TMB). Each experiment was conducted in triplicates, n=3.

FIG. 10 shows summary of number of visible spot (dot) counted in different concentration of agar as diffusion rate limiting medium (developing medium).

FIG. 11 shows summary of number of visible spot (dot) counted in different concentration of Carbopol 940 as diffusion rate limiting medium (developing medium).

FIG. 12 shows 100× magnification visible spot (dot) seen through the ELISA well using an upward looking microscope.

FIG. 13 summaries the result of the method of the invention using different diffusion rate limiting medium (developing medium) composition at the final stage of the assay.

FIG. 14 shows diagrammatic depiction of the non-immobilised form of the method of the invention. The CD27 positive cell is full coated with Rabbit anti-human CD27 antibodies followed by HRP conjugated goat anti-rabbit Fc region antibodies.

FIG. 15 shows photos taken with a macro ccd camera of A) microscopy slide containing 10 μL of CD27 labelled cells mixed with 0.028% Carbopol-ELISA specific TMB mix, B) microscopy slide containing 10 μL of CD27 labelled cells mixed with 0.028% Carbopol-ELISpot specific TMB mix, C) microscopy slide containing 10 μL of negative control mixed with 0.028% Carbopol-ELISA specific TMB mix and D) microscopy slide containing 10 μL of negative control mixed with 0.028% Carbopol-ELISpot specific TMB mix.

FIG. 16 shows photos taken from an upward looking microscope of A)×10 magnification of multiple visible spots (dots) formed on microscope slide and B)×100 magnification of a visible spot formed at the site of a target cell near a non-target cell showing no product formation by the lack of presence of colour.

FIG. 17 shows photos taken with a macro ccd camera of A) negative control where CD27 positive cells are immobilised with Rabbit anti-human CD27 antibodies and are not labelled with SARS-Cov-2 antigens and B) antigen-specific memory B cells are CD27 positive cells immobilised with Rabbit anti-human CD27 antibodies and labelled with SARS-Cov-2 Spike protein and nucleocapsid protein. Each experiment was conducted in triplicates, n=3.

FIG. 18 shows histogram representation of data from Table 1 of the negative control and the SARS-Cov-2-specific memory B cells expressed as dots/5×10⁵ whole PBMC.

### DESCRIPTION OF EMBODIMENTS

All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise” is generally used in the sense of include, that is to say permitting the presence of one or more features or components. Also as used in the specification and claims, the language “comprising” can include analogous embodiments described in terms of “consisting of” and/or “consisting essentially of”. The terms “including,” “comprising,” or “having,” and variations thereof, is meant to encompass the elements listed thereafter and equivalents thereof as well as additional elements. Embodiments recited as “including”, “comprising” or “having” certain elements are also contemplated as “consisting essentially of” and “consisting of” those certain elements.

As used in the specification and claims, the singular form “a”, “an” and “the” include plural references unless the context clearly dictates otherwise.

As used in the specification and claims, the term “and/or” used in a phrase such as “A and/or B” herein is intended to include “A and B”, “A or B”, “A”, and “B”.

The term “chromophore” as used herein refers to a label that changes colour in the visible spectra that can be observed without the aid of instrumentation.

The term “fluorophore” as used herein refers to a molecule with fluorescence properties. The fluorophore absorbs photons and emits photons of lower energy and visualisation requires the aid of fluorescence instrumentation.

The term “chemiluminescence” as used herein refers to the emission of light from a chemical reaction as a result of a substrate to product conversion. This product is in the excited state, while returning to ground state will emit light. Visualisation requires the aid of low-level light environment (e.g. dark room or dark box).

The term “reporter enzyme detection probe” as used herein comprises a reporter enzyme component comprising an enzymatic activity, coupled to a detection probe component comprising a target binding moiety. The reporter enzyme is optionally a peroxidase such as horseradish peroxidase or a phosphatase such as alkaline phosphatase although any stable enzyme that can produce ionizable products can be used including for example a lyase, hydrolase, synthase, synthetase, oxidoreductase, dehydrogenase, oxidase, transferase, isomerase, ligase, protease, such as trypsin, proteinase, peroxidase, glucose oxidase, myeloperoxidase, oxidase, monooxygenase, cytochrome, phosphatase such as alkaline phosphatase, decarboxylase, lipase, caspase, amylase, peptidase, transaminase, and kinase. Additional enzymes can include DNA or RNA polymerase, TAQ, restriction enzymes, klenow fragment, DNA ligase. The target binding moiety can either be a primary target binding moiety (e.g. a biopolymer such as an antibody or an antigen) that selectively bind a target substance, or be a secondary target binding moiety that selectively binds a primary detection agent. For example, the secondary target binding moiety can comprise a biopolymer such as an antibody or an antigen, that binds an antibody primary detection agent. Alternatively, the secondary target binding moiety comprises avidin or streptavidin that selectively binds a biotinylated primary detection agent, for example a biotinylated primary detection agent. In addition to antibodies and antibody binding fragments, other biopolymer target binding moieties that bind specifically to target substances or primary detection agents are also provided, including for example antibody mimetics, aptamers, binding polypeptides such as receptors as well as binding polypeptide mimetics, nucleic acids, carbohydrates and/or lipids. Examples include nucleic and/or peptide aptamers, affibodies and anticalins. For example, according to an embodiment, the primary antibody can be linked with enzymes, antigens, etc as long as the secondary antibody specific for the linked molecule and attached with an enzyme different from that of the primary antibody.

The term “primary detection agent” as used herein means an agent that selectively binds to a surface ligand of a target organism, a surface marker of a target organism or a target substance that is secreted by the target organism (e.g. a secretion marker of the target organism). The primary detection agent is optionally coupled to a detectable label such as biotin. In addition to antibodies and antibody binding fragments, other primary detection agents that bind specifically to surface ligands, surface markers, or target substances are also provided, including for example antibody mimetics, binding polypeptides, such as receptors, binding polypeptide mimetics, nucleic and peptide aptamers, affibodies and anticalins.

The present disclosure relates to a novel enzyme linked immunoassay using diffusion rate limiting medium at the last stage along with substrate to allow build-up of product concentration around the site of reaction and generate detectable dots from a target or targets of interest. Different combinations of capturing antibodies (optional); primary antibodies; enzyme probs (e.g. horseradish peroxidase, alkaline phosphatase etc.); substrates (e.g. chromophore, fluorophore, chemiluminescence and/or others); and vessels (e.g. 96-well plates, glass slips etc.); can be adapted into the system to detect both cell surface and cell secretion markers with a fraction of the setup cost compared to conventional methods. In addition, depending on the combination used, samples can be kept viable for other experiments and microscopy work can be carried out on clear vessels e.g. 96-well plates.

An aspect of the present invention provides a method for detecting a target organism in a sample, the method comprising;
a. optionally immobilizing the target organism to a solid phase;
b. incubating the target organism with one or more reporter enzyme detection probes to form one or more target organism-enzyme detection probe complexes;
c. removing any unbound reporter enzyme detection probes;
d. contacting the one or more target organism-enzyme detection probe complexes with a diffusion rate limiting medium containing one or more substrates and incubating the one or more target organism-enzyme detection probe complexes with the one or more substrates in the diffusion rate limiting medium to generate one or more detectable products in the diffusion rate limiting medium; and
e. detecting the one or more detectable products by detecting visible spots;
wherein the diffusion rate limiting medium is any suitable medium that decreases (limits) diffusion rate of the one or more detectable products, and thereby allowing formation of the visible spots.

In some embodiments, the step b) comprises incubating the target organism with one or more primary detection agents specific for the target organism prior to incubating with the one or more reporter enzyme detection probes to form the one or more target organism-enzyme detection probe complexes. In further embodiments, the one or more primary detection agents bind to one or more surface ligands of the target organism, one or more surface markers of the target organism or one or more target substances that are secreted by the target organism (e.g. a secretion marker of the target organism). In preferred embodiments, the one or more primary detection agents is an antibody or binding fragment thereof, or an antigen. In some embodiments, the one or more primary detection agents comprises biotin conjugated to an antibody or binding fragments thereof specific for the target organism or the target substance.

In some embodiments, the one or more surface markers of the target organism are selected from, but not limited to, the following lists:
B cells (target organism): CD27, CD19, CD21, CD20, CD38, CD23, CD138, IgA, IgG, IgM, B cell receptor.
T cells (target organism): CD4, CD8, T cell receptor, CD44, CD25, CD30.
Adult mesenchymal stem cells (target organism): CD10, CD13, CD73, CD105, CD271.
Embryonic stem cells (target organism): CD15, SSEA-3, CD324, CD90, CD117, CD29.

In some embodiments, the one or more target substances that are secreted by the target organism, is selected from, but not limited to, a group comprising:
B cells (target organism): IgA, IgG, IgM, INF-gamma, IL-6, IL-10, IL-13, lymphotoxins (TNF family cytokines).
T cells (target organism): IL-1, IL-4, IL-5, IL-6, IL9, IL-13, TGFβ.

In some embodiments, the step b) further comprises suppressing endogenous enzyme activity of the target organisms, such as human cells and E. coli. Suppressing endogenous enzyme activity of the target organisms is to reduce the possibility of background false positives (false dots) generated by endogenous enzyme activity of the target organisms. In a specific embodiment, if the target organism expresses endogenous enzymes (such as peroxidase) that are the same or similar (in activity) to the reporter enzymes of the one or more reporter enzyme detection probes, the step b) further comprises contacting the target organism with suppressors of said endogenous enzymes (such as a peroxidase suppressor). For example, the peroxidase suppressor is used to reduces background false positives (false dots) generated by endogenous peroxidase activity of some particular target organisms, such as human cells and E. coli.

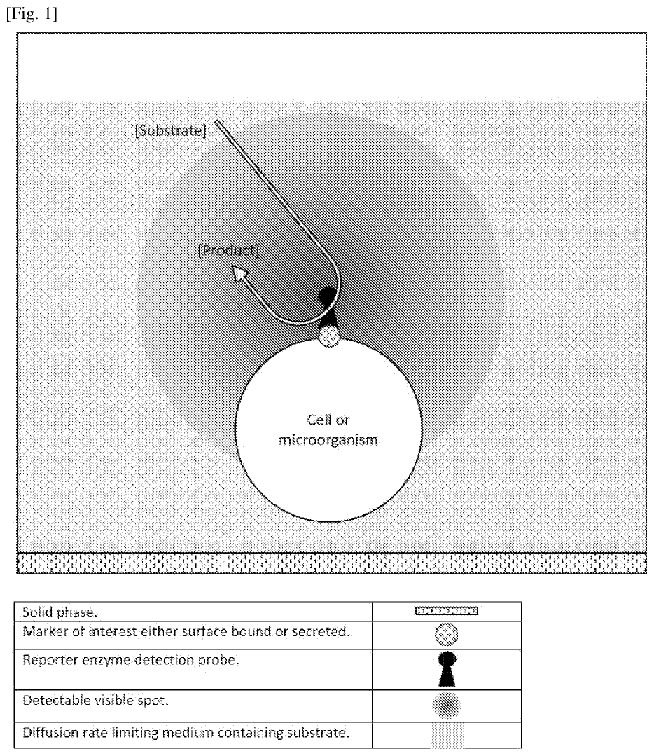

FIGS. 1 to 5 illustrate different configurations of the method of the invention. In [FIG. 1], the target organism with a specific marker (depicted in the example as a surface marker) is labelled with a marker specific reporter enzyme detection probe. This is mixed to form a suspension within a diffusion rate limiting medium with substrate specific to the reporter enzyme. Visible spots are produced as the reporter enzyme converts the substrate to the detectable product, creating an area of high concentration relative to the rest of the diffusion rate limiting medium, due to the detectable product diffusion being limited.

In some embodiments, the target organism is a cell or part thereof, or a microorganism or a part thereof. The target organism, such as the cell or the microorganism, can be living (alive) or dead. Preferably, if the target organism is not alive, the surface ligand or the surface marker of the target organism should be intact.

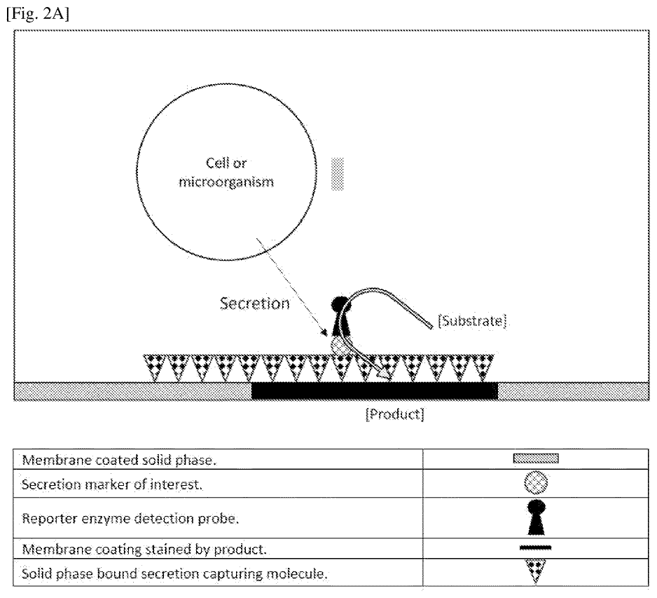

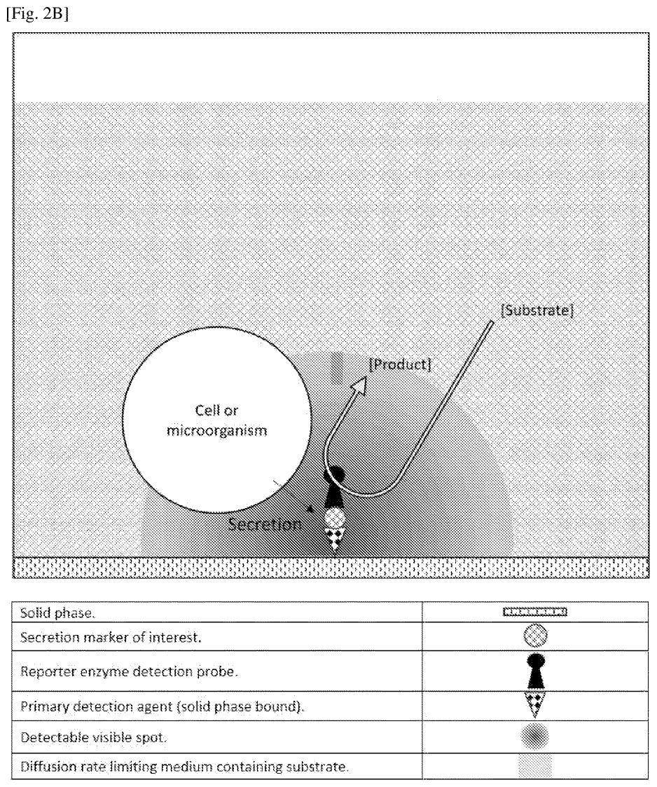

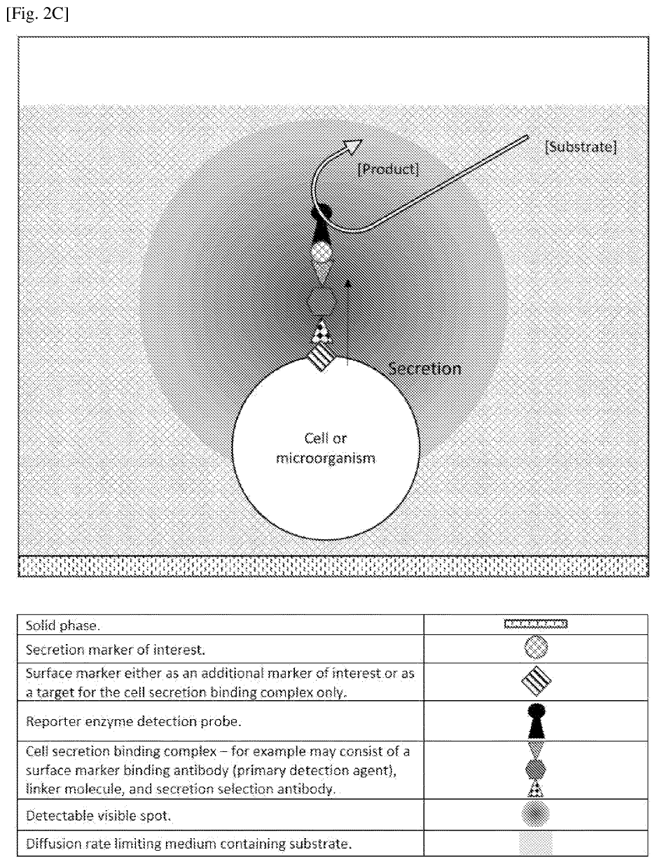

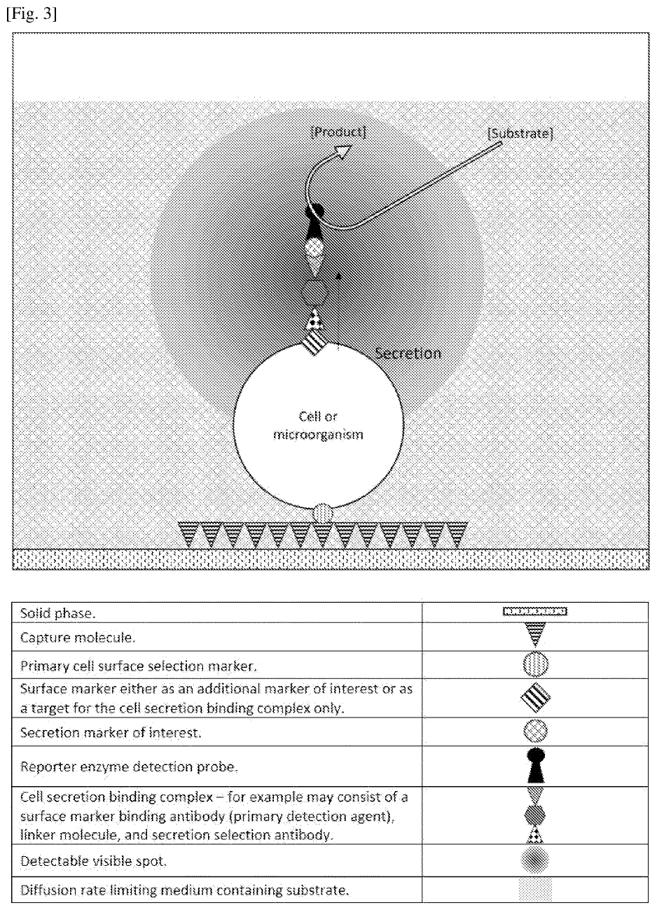

In some other embodiments, the detection of the target organism can be carried out by detecting a target substance that is secreted by the target organism. According to this embodiment, the target substance can act as a ligand for the reporter enzyme detection probe or for the primary detection agent. In some embodiments, the target substance is selected from the group comprising biopolymers, biomarkers, and proteins secreted by a cell or by a microorganism. In further embodiments, the target substance is selected from a group comprising a tumour marker, autoantigen, hormone, chemokine, cytokine, cardiac protein, a nucleic acid molecule, lipid, and carbohydrate. Typically, the method according to the present invention for detecting a target substance that is secreted by the target organism can be carried out as follows: the primary detection agent sticks onto the cell surface for first selection, followed by a scaffolding system, e.g. biotin-streptavidin, to link the target substance capturing antibody to primary detection agent, allow the secretion of the target substance to occur, then tag the other end of the target substance with the reporter enzyme detection probe (see [FIG. 2]). Optionally, the cells that secrete the target substance, can be immobilized on a solid phase as disclosed below (see [FIG. 3]).

In some embodiments, the sample is a body fluid, a body tissue or any material or composition susceptible to contain the target organism, such as cells or microorganisms, preferably a sample is a blood sample.

In some embodiments, the target organism is immobilized by directly binding the solid phase, optionally by adsorption to the solid phase, or is immobilized to the solid phase indirectly by a capture molecule coupled to the solid phase that binds the target organism. The capture molecule, which is coupled to the solid phase, binds to the same or different surface ligand or surface marker of the target organism than the one or more reporter enzyme detection probes or the one or more primary detection agents. In preferred embodiments, the capture molecule is an antibody or binding fragment thereof, an antigen or a ligand. For example, the capture molecule is anti-CD27 antibody. The capture molecule will typically immobilize T cells, NK cells and memory B cells, plasmablast B cells and plasma B cells from the blood sample on the plate. Upon all the cell types mentioned above, only memory B cells and plasmablast B cells express B cell receptors and directly binds to the reporter enzyme detection probe or to the primary detection agent. In some embodiments, the capture molecule allows selection of (specifically targeting) the target organism.

In some embodiments, the solid phase is a reaction vessel, a bead, a platform or a plate. Example reaction vessels can be selected from a group comprising 96-wells plate, 6 wells plate, a petri dish, and tissue suitable tube such as a falcon tube. In other embodiments, the surface of the solid phase is selected from metal, gold, stainless steel, plastic, glass, silica, polycarbonate, polyester, PVDF, polystyrene, nitrocellulose, and cellulose.

In some embodiments, the incubation in step b) is carried out in a solution under conditions to form the one or more target organism-enzyme detection probe complexes. The solution used for incubation in step b) can be selected from, but not limited to, a group comprising RPMI1640+10% Foetal Calf Serum, DMEM, HEPES, MEM, DMEM F12, IMDM, M199, Ham's F12, Ham's F10, HPLM, foetal bovine serum, and Human serum. The incubation conditions in step b) are typically 30 minutes to 4 hours, preferably 1 hour to 3 hours; and the temperature can range from 20° C. to 40° C.; preferably 25° C. to 40° C., most preferably 35° C. to 40° C.

The one or more reporter enzyme detection probes bind to one or more surface ligands of the target organism, one or more surface markers of the target organism, one or more target substances that are secreted by the target organism or the one or more primary detection agents. In other embodiments, the reporter enzyme detection probe is selected from antibodies or binding fragments thereof, antigens, drugs, or peptides, which are directly or indirectly conjugated to an enzyme or a catalyst that can convert a colour in contact with the substrate.

In an embodiment, the reporter enzyme detection probe comprises a primary target binding moiety (e.g. for direct target organism binding or direct target substance binding), or a secondary target binding moiety (e.g. for indirect target organism binding or indirect target substance binding) and the reporter enzyme comprising enzymatic activity, wherein the target binding moiety is covalently bound to the reporter enzyme.

In another embodiment, the reporter enzyme is or comprises lyase, hydrolase, synthase, synthetase, oxidoreductase, dehydrogenase, oxidase, transferase, isomerase, ligase, protease, such as trypsin, proteinase, peroxidase, glucose oxidase, myeloperoxidase, oxidase, monooxygenase, cytochrome, alkaline phosphatase, decarboxylase, lipase, caspase, amylase, peptidase, transaminase, and/or kinase activity. In another embodiment, the reporter enzyme is selected from DNA or RNA polymerase, TAQ, restriction enzymes, klenow fragment and DNA ligase. In preferred embodiment, the reporter enzyme is or comprises horseradish peroxidase or alkaline phosphatase.

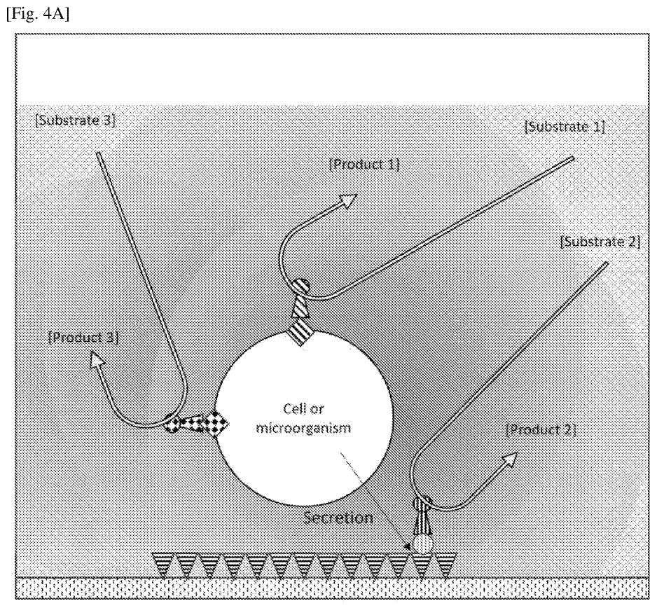

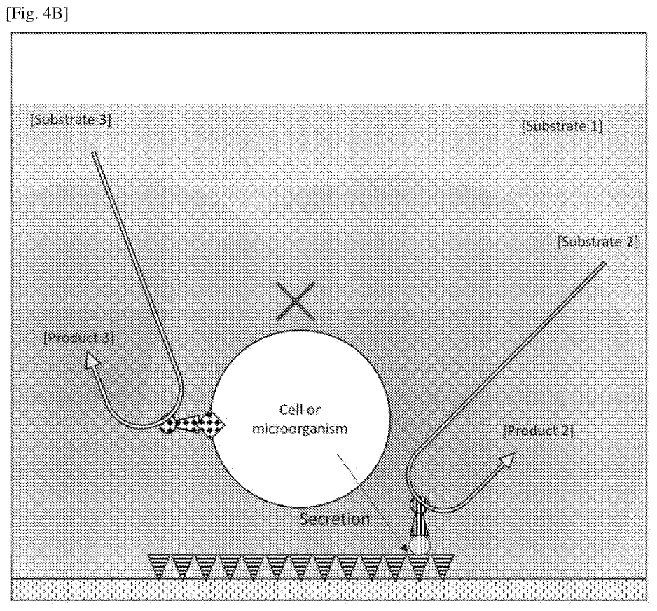

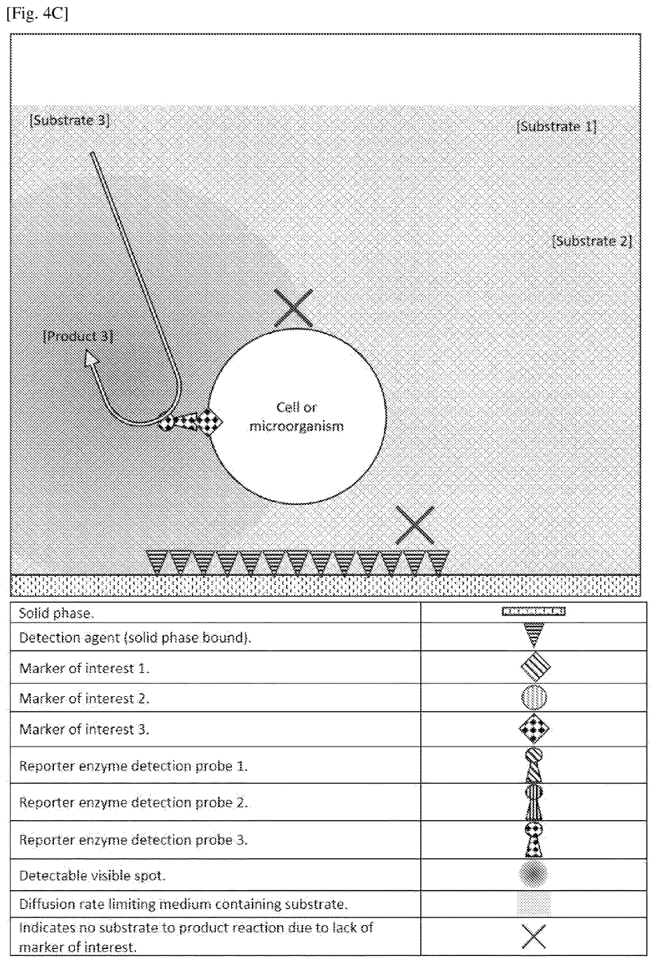

In some embodiments, the reporter enzyme detection probes are different, namely each reporter enzyme detection probe being specific for one specific marker, i.e. one specific surface marker, one specific surface ligand or one specific secretion marker (see for example [FIG. 4]). In some embodiments, the use of more than one different reporter enzyme detection probes allows targeting one target organism having different markers (surface markers, surface ligands and/or secretion markers). In some other embodiments, the use of more than one different reporter enzyme detection probes allows targeting different target organisms, having different markers (surface markers, surface ligands and/or secretion markers). Thus according to an aspect, the method of the invention allows detecting one or more target organisms in a sample the method comprising
a. optionally immobilizing the one or more target organisms to a solid phase;
b. incubating the one or more target organisms with one or more reporter enzyme detection probes to form one or more target organism-enzyme detection probe complexes;
c. removing any unbound reporter enzyme detection probes;
d. contacting the one or more target organism-enzyme detection probe complexes with a diffusion rate limiting medium containing one or more substrates and incubating the one or more target organism-enzyme detection probe complexes with the one or more substrates in the diffusion rate limiting medium to generate one or more detectable products; and
e. detecting the one or more detectable products by detecting visible spots;
wherein the diffusion rate limiting medium is any suitable medium that decreases diffusion rate of the one or more detectable products and thereby allowing formation of the visible spots.

In some embodiments, removing any unbound reporter enzyme detection probes is carried out by washing the solid phase.

According to a specific embodiment, when the target organism is not immobilized to a solid phase, removing any unbound reporter enzyme detection probe is carried out by centrifugation. Typically, the washing is conducted in a vessel, such as tissue culture grade sterile test tubes (e.g. 15 mL falcon tubes). The target organisms, such as cells, (with a higher density) will sediment (pellet out) by centrifugal force and the unbound reporter enzyme detection probes will remain in solution. The supernatant (containing the unbound reporter enzyme detection probes) is decanted and fresh medium is added to further dilute unbound reporter enzyme detection probes. The pellet cells are resuspended and the process repeated×3. At the end, the labelled and unlabelled target organisms, such as cells, remain in a medium. The unbound reporter enzyme detection probes are decanted off.

The substrate is a molecule that is catalysed by the reporter enzyme to provide a chromogenic detectable product, a fluorogenic detectable product or chemiluminescent detectable product. The detectable products provide visible spots. In some embodiments, the substrate is selected from, but not limited to, the group comprising tetramethylbenzidine (TMB), 5-bromo-4-chloro-3-indolyl-beta-D-galacto-pyranoside (X-Gal), 5-bromo-4-chloro-3-indolyl phosphate (BCIP), p-nitrophenol (PNPP), 3,3′-Diaminobenzidine (DAB), 4-(Trifluoromethyl) umbelliferyl phosphate, 4-Methylumbelliferyl β-D-galactopyranoside (Mu-Gal), adamantyl 1,2-dioxetane phosphate, o-nitrophenyl-β-D-galactopyranoside (ONPG), Nitro blue tetrazolium (NBT), 2,2′-Azinobis [3-ethylbenzothiazoline-6-sulfonic acid]-diammonium salt (ABTS), o-phenylenediamine dihydrochloride (OPD), Nitro blue tetrazolium chloride (NBT), N-Acetyl-Leu-Glu-His-Asp-7-amino-4-Trifluoromethylcoumarin, N-Acetyl-Leu-Glu-Thr-Asp-7-amino-4-Trifluoromethylcoumarin, and 3-oxo-3H-phenoxazin-7-yl-butanoic acid ester (Resorufin butyrate).

One or more same or different substrates can be used according to the method of present invention. Typically, more than one different substrate is used when more than one different reporter enzyme detection probes are used. Markers of the target organism are targeted so that they can be differentiated. The use of more than one substrate allows detecting one target organism having different markers (surface markers, surface ligands and/or secretion markers) or detecting different target organisms, having different markers (surface markers, surface ligands and/or secretion markers).

TABLE 1
Non-exhaustive examples of substrates

| | | Color (Before |
|---|---|---|
| | | conversion/After |
| Enzyme | Substrate | conversion) |
| β-galactosidase | 5-bromo-4-chloro-3-indolyl β-D-galactopyranoside | Clear/dark blue p |
| | (X-Gal or BCIG) | recipitate |
| | o-nitrophenyl-β-D-galactopyranoside (ONPG) | Clear/Yellow |
| Alkaline | 5-Bromo-4-chloro-3-indolyl phosphate (BCIP)/Nitro | Clear/Blue to |
| Phosphatase | blue tetrazolium (NBT) | purple |
| | p-Nitrophenyl Phosphate(PNPP) | Clear/Yellow |
| Horse Radish | 3,3′,5,5′-tetramethylbenzidine (TMB) | Clear/Blue |
| Peroxidase | 2,2′-Azinobis [3-ethylbenzothiazoline-6-sulfonic acid]- | Clear/Green |
| | diammonium salt (ABTS) | |
| | o-phenylenediamine dihydrochloride (OPD) | Clear/Yellow to o |
| | | range |
| | 3,3′-diaminobenzidine (DAB) | Brown |
| | Metal-enhanced DAB | Brown to black |
| Glucose oxidase | Nitro blue tetrazolium chloride (NBT) | |
| Caspase-4, 5, 9 | N-Acetyl-Leu-Glu-His-Asp-7-amino-4- | Fluorescent |
| | Trifluoromethylcoumarin | |
| Caspase 8 | N-Acetyl-Leu-Glu-Thr-Asp-7-amino-4- | Fluorescent |
| | Trifluoromethylcoumarin | |
| Triglyceride | 3-oxo-3H-phenoxazin-7-yl-butanoic acid ester | Fluorescent |
| lipases and | (Resorufin butyrate) | |
| cholineste rases | | |

The diffusion rate limiting medium (DRLM) is designed to host the substrate (that ultimately generates visible spots upon catalysis by the reporter enzyme of the reporter enzyme detection probe); to be porous in order to allow flow of unconverted substrate and converted detectable product (i.e. before and after catalysis by the reporter enzyme); to slow down diffusion rate of converted substrate concentrating the product into the detectable product that allows formation of visible spots around the site of the reporter enzyme probe reaction site; to provide an interface for the reaction between the reporter enzyme, part of the reporter enzyme detection probe, which is bound to the target organism and the substrate. The diffusion rate limiting medium (DRLM) is any suitable medium that limits diffusion rate either by physical property, for example pore size, or by chemical properties such as charge, hydrophobicity, pH, etc.

Contrary to ELISpot and ELISA, wherein the enzyme converted product gets absorbed in the membrane of an ELISpot well forming stained spots (See [FIG. 2A]) or ELISA which will form coloration in the whole solution, the method of the present invention uses a diffusion rate limiting medium to slow down the diffusion of the product and trapping it around the cell that has been marked by the reporter enzyme detection probe and thereby allowing formation of detectable dots within the diffusion rate limiting medium (DRLM).

In some embodiments, the diffusion rate limiting medium (DRLM) may also provide environment for the target organism viability, for example providing osmolarity regulation and pH buffering and nutrients.

In further embodiments, the diffusion rate limiting medium (DRLM) is non-toxic (for example for non-endpoint applications).

The diffusion rate limiting medium (DRLM), that decreases diffusion rate of the detectable product may have different positions in respect to the target organism. Namely, the target organism can be above the DRLM ([FIG. 5A]), under the DRLM ([FIG. 5B]) or within the DRLM ([FIG. 1]).

In some embodiments, the diffusion rate limiting medium is a semi-solid medium, a membrane or a viscous fluid. According to an embodiment, the semi-solid medium is selected from the group comprising agar, agarose, hydrogel (e.g. Carbopol 940, 2NapFF), gelatine, silicone gel, cellulose derivatives, and SDS-PAGE gel. In another embodiment, the membrane is selected from a group comprising a paper membrane, a fiber membrane, a cellulose membrane, and a plastic membrane. In a further embodiment, the viscous fluid is selected from a group comprising a cellulose derivative (carboxymethylcellulose, etc), a polysaccharide or monosaccharide gel (sugar syrup, low concentration hydrogel, cassava gel, guar gum, gellan gum, xanthan gum, acacia gum, hydroxyethylcellulose, HPC (hydroxypropyl cellulose)), and a low concentration hydrogel.

In some embodiments, the detectable product is a molecule that is chromogenic, chemiluminescent or fluorescent. In further embodiments, the detectable product can have radiological and/or electrochemical properties.

The visible spots can be detected manually with a microscope or automatically with a suitable apparatus, such as a macro camera. If the visible spots are outside the visible light spectrum that the human eye can view, than the visible spots are detected automatically with a suitable apparatus, such as a macro camera, adapted for such light spectrum. In case of chromogenic data, typically a picture can be taken of individual wells on 96-well plates with an apparatus, such as a macro camera, and with software visible spots can be quantified. If chemiluminescence or fluorescence is used, specialised filters and lamps for fluorescence and chemiluminescence can be used to detect visible spots and quantify them.

The size and formation of the visible spots are governed by diffusion rate of the substrate and/or the detectable product. The diffusion rate is typically affected by physical properties of the diffusion rate limiting medium (DRLM) such as porousness; chemical properties such as charge or pH; incubation temperature, which can be important for the reporter enzymes, and where the higher temperature provides faster the diffusion rate; duration of the incubation time; chemical and physical properties of the diffusion rate limiting medium (DRLM) and the substrate (as well their interactions); the reporter enzyme kinetics (the reporter enzyme speed in converting substrate and thereby formation of visible spots) and available concentration of substrate to feed the reaction. In preferred embodiments, a high concentration of the detectable product is formed around the target organism trapped by the slow diffusion rate and forms the visible spots.

The selection of suitable diffusion rate limiting medium (DRLM) can be carried out as follows. Literature search and or experimental tests must be conducted to determine the suitability of a diffusion rate limiting medium (DRLM). The following key functions of the diffusion rate limiting medium should be taken into consideration:
1. The ability to host the substrate.
2. The porousness and/or other chemical and/or physical properties which might affect diffusion of substrates to the enzyme reaction location and of detectable products away.
3. The ability to manipulate the diffusion rate limiting medium properties to affect diffusion rate of products to allow concentration build up around the enzyme to form detectable visible spots.
4. Provide an interface for enzymes to react with the substrate.
5. Non-inhibiting to the reporter enzyme/substrate reaction.
6. For non-end-point assay, the diffusion rate limiting medium should be non-toxic, and provide conditions to maintain viability, including pH, osmolarity and nutrients, for targeted samples.

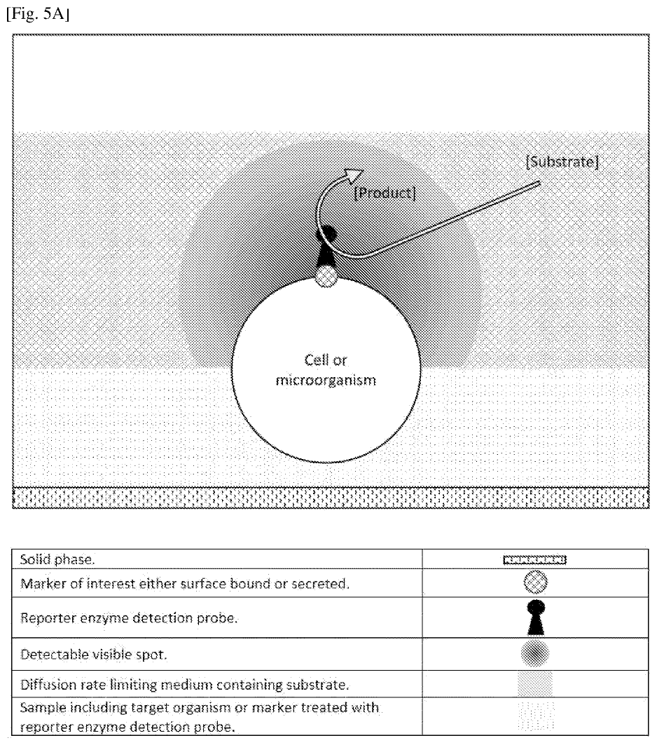

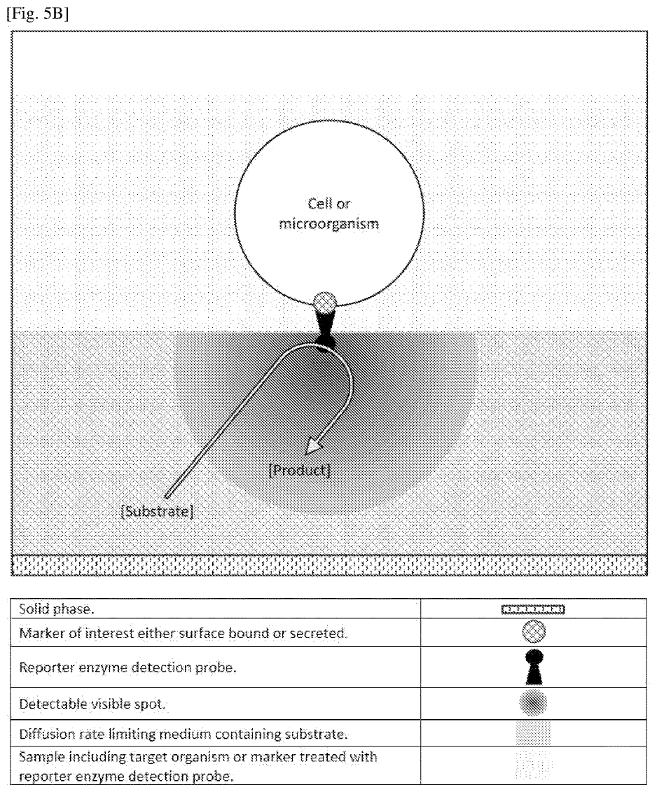

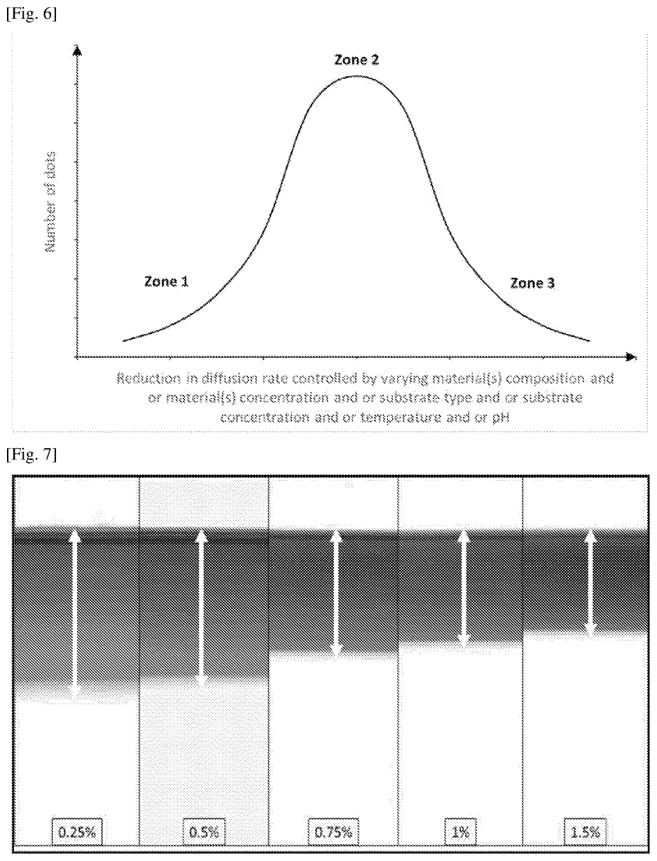

After selecting the material(s), optimal diffusion rate limiting medium composition and concentration tests should be conducted. This should be conducted by preparing a series of compositions and concentrations of the material(s), that make up the diffusion rate limiting medium, prepared in diluent and substrate. Examples of diluent are tissue culture media and or phosphate buffer saline (PBS) and or solvents that are compatible with the material(s), sample types and substrate. The choice of substrate is specific for the reporter enzyme conjugated on the detection probe. A positive control reporter enzyme detection probe, specific for the sample type common surface marker, should be used to assess the optimal diffusion rate limiting medium according to the flowing variables; material(s) composition, material(s) concentration, substrate type, substrate concentration, temperature, pH and time (other variables should be considered but may be application specific. A series of optimisation steps should give results that appear similar to the diagram depicted in [FIG. 6]. The optimal material(s) composition and concentration should generate the maximum number of visible spots, as described in zone 2 of [FIG. 6].

FIG. 6 shows diagrammatic depiction of number of dots (y-axis) vs diffusion rate high to low of substrate and product as controlled by DRLM properties (x-axis). The diagram is divided into 3 zones. Zone 1 shows low dot counts where the DRLM properties are insufficient at limiting the diffusion of the substrate/product. This would turn the medium a uniform colour with very few detectable visible spots or in some cases random nonspecific product precipitation with uncountable results. Zone 2 shows optimal DRLM properties and results should show a medium with little to no coloration with high visible spot counts. Zone 3 shows low visible spot counts when the DRLM properties are limiting diffusion too much, thus limiting the diffusion of the substrate/detectable product. The results of such tests would expect to show little enzyme activity so a declining number of visible dots and very little or no medium colouration.

Variables effecting diffusion of substrate and product are, but not limited, to physical and chemical properties of the DRLM, variance in the composition, and concentration of DRLM materials, substrate type and concentration, product type and concentration, incubation temperature, and pH. Changes of each variable might expect to generate a graph as depicted in [FIG. 6]. Zone 1 shows typical results where, the DRLM is insufficiently limiting diffusion rate leading to rapid diffusion of the product into the bulk medium. This result is typified by a medium which undergoes colour change, and less than expected clear countable detectable visible spots and in some cases random precipitation of product. Zone 2 shows what might be expected where optimal conditions are met for the DRLM to form clear detectable visible spots in the highest quantities. Zone 3 shows the scenario where DRLM and conditions are limiting diffusion beyond optimal thus limiting detectable visible spot formation. A reduction of detectable visible spots may be the result of insufficient substrate availability, diffusion of the product so slow it remains with in microscopic limits at the point of reaction or other factors such as unexpected enzyme inhibition for example.

Considerations for duration of incubation for visible spot formation must allow for optimal numbers of visible spots (dots) to be formed at a detectable size/volume and not be so long such that visible spots diffuse to a point of being undetectable. Incubation temperature should be considered also that too low may reduce enzyme activity in such a way to limit formation of visible spots. Conversely too high may prevent or denature the enzyme such that the reaction cannot occur thus resulting also in reduced formation of visible spots.

In an embodiment, any of the antibodies disclosed herein may be a monoclonal antibody, polyclonal antibody, chimeric antibody, and/or monospecific antibody.

In some embodiments, the incubation step d) is carried out between 20° C. to 40° C., preferably 23° C. to 37° C. The incubation time is at least 30 minutes, preferably 30-45 minutes.

Another aspect of the present invention provides a method of quantifying the amount of a target organism in a sample, the method comprising
detecting the target organism according to the method of the invention disclosed herein; and
quantifying the amount of the target organism in the sample based on the number of the visible spots.

Another aspect of the present invention provides a method for selecting and isolating a target organism in a sample containing diverse organisms, the method comprising
detecting the target organism according to the method of the invention disclosed herein; and
isolating the target organism.

Once the target organism is detected by the method of the invention, it can be isolated by methods known in the art, such as pipetting.

The method of the invention for detecting a target organism (or one or more target organisms) in a sample has many advantages compared to ELISpot. For example, the method of the invention uses a B-cell specific surface marker as target for the capturing antibodies (the capture molecule) to immobilise the cells on the solid phase, then uses the specific antigen binding mechanism of B cell for the reporter enzyme detection probe. By such procedure, the targeted B-cells of interest can be isolated, and by targeting the antigen binding rather than a secretion marker, there is no need to wait for cell differentiation and secretion. This results in shortening a traditional 7-day B cell assay to 1 day and a true B-cell count is provided by direct detection rather than indirectly through secretion or after cell differentiation. Another advantage is that the mechanism of obtaining visible spots (i.e. converting the colour) is carried out in the DRLM and not at the platform surface as for ELISpot, limiting its use to secretion markers only and requiring more expensive membrane coated platforms and unlike ELISA it can isolate and quantify cells. A further advantage is that the platforms used can be clear allowing microscopy study and DRLM can be designed to maintain cell visibility. Moreover the present invention enables multi marker detection (multiplex) and cell isolation to be carried out on the single same group of cells in a single assay. Thus, for example, the method of the invention can replace the following in one go: FACS to profile cell surface marker in one cell group, ELISpot or ELISA to detect secretion marker in another cell group, then returning to the cells from FACS's cell sorting to dilute it out for cloning.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended claims, and all changes which come within the meaning and range of equivalency are intended to be embraced therein.

The foregoing description will be more fully understood with reference to the following Examples. Such Examples, are, however, exemplary of methods of practising the present invention and are not intended to limit the application and the scope of the invention.

### EXAMPLES

1-Concentration of the diffusion rate limiting medium affecting diffusion rate This is a sample text. This example shows how this invention can be helpful.

### Aim:

The aim of this experiment is to demonstrate how concentration of the diffusion rate limiting medium affects diffusion rate using agar (a possible diffusion rate limiting medium (DRLM) candidate) and methylene blue (the diffusing agent) as a model.

### Materials:

Agar and methylene blue were purchased from Sigma-Aldrich. Isopropyl alcohol (IPA) was purchased from Anaqua. Type 1 water was generated from Merck Direct-Q® 5 UV. 25° C. and 50% non-condensing humidity was maintained using a climate control cabinet from MRC lab. The macro ccd camera that was used to take the image was purchased from sh-renyue including calibration software S-eye. Data processing software, Icy software including Image J, was downloaded from https:/icy.bioimage analysis.org/. SDS gel casting apparatus was purchased from Bio-Rad.

### Method:

4 mL of agar gel in the following compositions, 0.25% (W/V), 0.5% (W/V), 0.75% (W/V), 1% (W/V) and 1.5% (W/V) in type 1 water were prepared and added to SDS gel casting apparatus. A layer of IPA was added on top of the gel as a levelling agent and the gel allowed 30 mins to set. The IPA was then poured away and washed thoroughly with water. The top of the gel was then dabbed dry by sliding lint free tissue between the glass slides. 200 μL Methylene blue was added on top of the gel, and the apparatus placed inside a climate control cabinet at 25° C. with 50% non-condensing humidity for 1 hour. After 1 hour, the gel was removed from the climate control cabinet and the remaining methylene blue was rinsed away with water and dabbed dry as previously done. Images of each gel were taken using a stationary mounted macro-camera. A pixel to mm calibration was performed using an ISO certified calibrated ruler and the distance of diffusion of methylene blue after 1 hour calculated by pixel number using ImageJ software.

### Result

All pictures were taken using S-eye software under fixed settings and saved in jpg format. ImageJ was used to determine the pixel/mm correlations against a calibrated ruler. Five calibration points were taken and plotted as Pixel vs length (mm). The best fit line (trendline) with an R²=1 with an equation: (y (pixel)+8.073)/341.7=x (length, mm). The equation was then used to calculate the distance of methylene blue traveled.

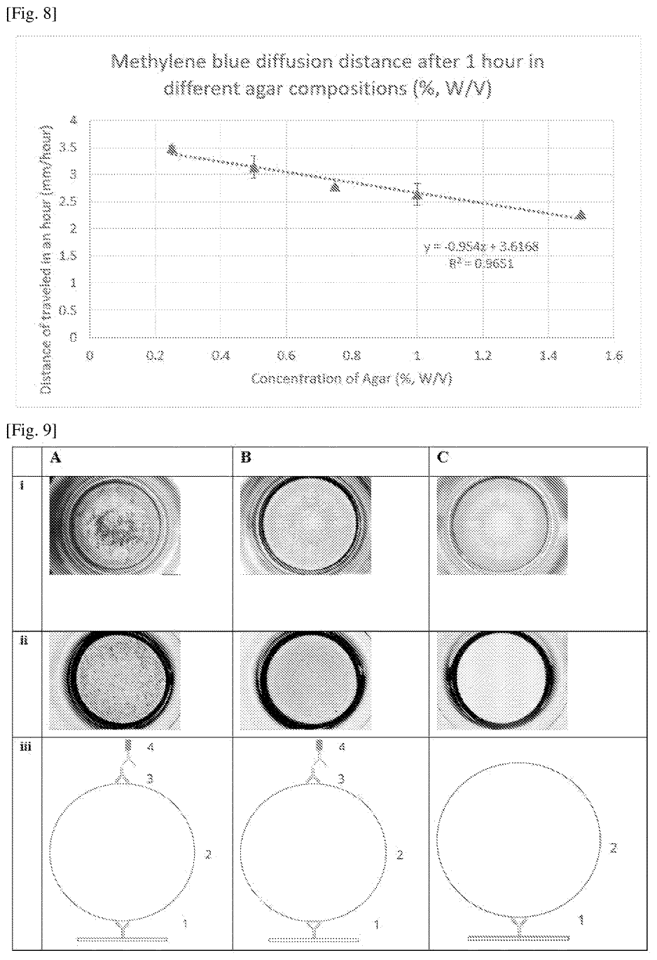

The distance diffused in 1 hour by the methylene blue in different agar compositions (0.25% (W/V), 0.5% (W/V), 0.75% (W/V), 1% (W/V) and 1.5% (W/V)), was determined by measuring the pixel length from the top of the agar to the methylene blue diffusion front (see [FIG. 7]). Each agar composition was repeated three times, and five measurements were taken from each repeat. The distance diffused in one hour of each agar composition are plotted in [FIG. 8]. The best fit line (trendline) with an R²=0.97 (coefficient of determination, R²>90% or 0.9, very strong to perfect relationship) agrees with the linear regression model with an equation: y (diffusion rate in mm/hour)=−0.954x (agar composition % (W/V))+3.6168. With increase in agar composition (%, W/V) there is a decrease in distance diffused by methylene blue 1 hour indicating a slower diffusion rate (mm/hour).

### Conclusion

The diffusion distance of methylene blue after 1 hour decreases proportionally as the concentration of agar (%, W/V) increases and vice versa. This provides evidence that as the diffusion rate limiting medium concentration increases, diffusion rate within the diffusion rate limiting medium decreases.

### 2—Presence and Absence of the Diffusion Rate Limiting Medium

### Aim:

To prove that clear defined dots (visible spots) are only formed while a suitable diffusion rate limiting medium (DRLM) is present at the last step of the assay to slow down the diffusion rate of the substrate and the detectable product regardless of the vessel.

### Materials

Rabbit anti-human CD27 antibodies, HRP conjugated goat anti-rabbit Fc region antibodies and Lymphoprep were purchased from Abcam. Phosphate buffer saline (PBS), Foetal Calf Serum (FCS), RPMI 1640 with phenol red, Ethylenediaminete traacetic acid (EDTA), sodium bicarbonate (NaHCO₃) and Peroxidase suppressor was purchased from ThermoFisher Scientific. ELISpot plates, trypan blue solution, RPMI 1640 without phenol red and agar was purchased from Merck/Sigma-Aldrich. ELISA plates were purchased from Jet Biofil. 3,3′,5,5′-Tetramethylbenzidine (TMB) was purchased from Mabtech. Agarose was purchased from Bio Basic.

The macro ccd camera that was used to take the image was purchased from sh-renyue including calibration software S-eye. Data processing software, Icy software including Image J, was downloaded from https:/icy.bioimageanalysis.org/.

### Method

ELISA and ELISpot plates were coated with Rabbit anti-human CD27 antibodies (0.5 μg/50 μL/well), made up in phosphate buffer saline (PBS) at pH7, minimum 12 hours before the experiment. The wells were then washed with PBS six times and blocked with RPMI 1640 containing 10% FCS.

On the day of the experiment, fresh blood samples were taken from volunteers. The blood was immediately diluted (50:50) in RPMI 1640 containing 1 mg/mL EDTA. Note: RPMI 1640 for cell culture work was conducted with RPMI 1640 with phenol red. Lymphocyte separation was conducted by taking the diluted blood and layered on top of 10 mL of lymphoprep, which was then centrifuged at 800 g for 30 mins with the brakes off. The lymphocyte layer was extracted using a sterile disposable pipette and fresh RPMI 1640+10% FCS+1 mg/mL EDTA+0.2% NaHCO₃ was added to dilute the lymphocyte containing lymphoprep. The lymphocyte was subjected to centrifugation at 300 g for 10 mins. The supernatant was removed and fresh RPMI 1640+10% FCS+1 mg/mL EDTA+0.2% NaHCO₃ was added, which was then centrifuged at 300 g for 10 mins. This cleaning process was repeated 3 times.

Lymphocytes were then resuspended RPMI 1640+10% FCS+1 mg/mL EDTA+0.2% NaHCO₃. 100 μL of the resuspended cells were taken and diluted in 900 μL RPMI 1640+10% FCS+1 mg/mL EDTA+0.2% NaHCO₃ (1 in 10 dilution). The cells were vortexed and 100 μL of this cell suspension was taken and added to 100 μL of trypan blue solution, which was then vortexed. 20 μL of trypan blue suspended cells were added onto a Neubauer improved counting chamber and cells were counted.

5×105 cells/well was seeded in RPMI 1640+10% FCS+1 mg/mL EDTA+0.2% NaHCO₃ and incubated at 37° C. for 2 hours. After 2 hours, the wells were washed six times in warm (37° C.) PBS. Positive CD27 cells were further labelled with Rabbit anti-human CD27 antibodies (0.5 μg/50 μL/well) prepared in RPMI 1640+10% FCS+1 mg/mL EDTA+0.2% NaHCO₃. Negative control wells were given RPMI 1640+10% FCS+1 mg/mL EDTA+0.2% NaHCO₃ only. The plates were then incubated at 37° C. for 2 hours. After 2 hours, the wells were washed six times in warm (37° C.) PBS.

The wells were treated with peroxidase suppressor for 15 mins at 4° C. followed by 10 mins at room temperature (RT). After peroxidase suppressor treatment, the wells were washed six times in warm (37° C.) PBS. HRP conjugated goat anti-rabbit Fc region antibodies, prepared 1 in 1000 dilution in RPMI 1640+10% FCS+1 mg/mL EDTA+0.2% NaHCO₃, were added to the Rabbit anti-human CD27 antibodies labelled wells and RPMI 1640+10% FCS+1 mg/mL EDTA+0.2% NaHCO₃ were added to the negative control wells. The plates were left to incubate at RT for 1 hour. After 1 hour, the wells were washed six times in warm (37° C.) PBS.

Wells without the diffusion rate limiting medium were prepared by taking stock TMB solution and mixed with equal volume of 2×RPMI 1640 without phenol red. 40 μL of the TMB/RPMI 1640 mix was added to each well. Note: RPMI 1640 used at this at this stage of the work does not contain phenol red. Wells with semi-solid medium were prepared by taking 1:1:2 ratio of 4×RPMI: 1.2% Agar (or agarose)—warmed to 80° C.: stock TMB solution leading to a 0.3% agar (or agarose) TMB semi-solid medium. 40 μL of the semi-solid TMB medium was added to each well. The plates were then incubated at 37° C. for 30 mins. Photos were taken using a macro ccd camera.

### Results

Wells without the diffusion rate limiting medium (such as a semi-solid medium) on both [FIG. 9], Ai) ELISpot and Aii) ELISA plate platforms yielded clumpy precipitants in solution. No clear definable dots were formed by the cells thus no quantifiable data was obtainable. Note: the TMB solution that was used in this experiment was conducted using ELISpot specific TMB which form precipitation.

Wells with the diffusion rate limiting medium on both [FIG. 9] Bi) ELISpot and Bii) ELISA plate platforms yielded countable and visible spots (dots). By reducing the diffusion rate of the TMB as the reaction occurs, the precipitant is concentrated locally by the cell where the reaction occurred, thus forming the colour and visible spots (dots). 0.3% agar/RPMI/TMB diffusion rate limiting medium (developing medium) was used for the ELISpot plates (See [FIG. 9] Bi). Whereas 0.3% agarose/RPMI/TMB diffusion rate limiting medium (developing medium) was used for the ELISA plates (See [FIG. 9] Bii). Visible spots (dots) were formed in both plates while using either low concentrations of agar or agarose as the base of the diffusion rate limiting medium (developing medium). This has demonstrated that the method of the invention can be adapted to multiple vessels and diffusion rate slowing down medium.

Wells with the diffusion rate limiting medium on both [FIG. 9] Ci) ELISpot and Cii) ELISA plate platforms, negative controls, yielded very few to no visible spots (dots). These negative controls show that the absence of Rabbit anti-human CD27 antibodies and HRP conjugated goat anti-rabbit Fc region antibodies labelling show few to no visible spots (background noise). More importantly, the presence of the cells with the specific target markers that are eliciting the formation of the blue dye seen the above-mentioned examples show distinct visible spots (dots).

### Conclusion

The result in both ELISpot and ELISA plate platforms detectable and quantifiable spots are only formed in the presence of a suitable DRLM. In both cases if no suitable DRLM is used the experiment will fail to produce quantifiable data.

### 3—Visible Spots Formation and Diffusion Rate

### Aim:

To prove that visible spots formation depends on the diffusion rate.

### Materials:

Rabbit anti-human CD27 antibodies, HRP conjugated goat anti-rabbit Fc region antibodies and Lymphoprep were purchased from Abcam. Phosphate buffer saline (PBS), Foetal Calf Serum (FCS), RPMI 1640 with phenol red, Ethylenediaminete traacetic acid (EDTA), sodium bicarbonate (NaHCO₃) and Peroxidase suppressor were purchased from ThermoFisher Scientific. ELISpot plates, trypan blue solution, RPMI 1640 without phenol red and agar were purchased from Merck/Sigma-Aldrich. ELISA plates were purchased from Jet Biofil. 3,3′,5,5′-Tetramethylbenzidine (TMB) was purchased from Mabtech. Carbopol940, sodium hydroxide (NaOH) and glycerol were purchased from Acros Organics. Type 1 water was generated from Merck Direct-Q® 5 UV.

The macro ccd camera that was used to take the image was purchased from sh-renyue including calibration software S-eye. Data processing software, Icy software including Image J, was downloaded from https:/icy.bioimageanalysis.org/.

### Method

0.056% Carbopol 940 was prepared by taking 75 mg of Carbopol 940 into 100 mL room temperature pre-boiled ultrapure water. Using a magnetic stirrer, allow to mix at maximum speed overnight for the wetting process. Titrate and record the volume of 0.1M NaOH added to the mixture until reaching pH7.0 (33 mL of 0.1M NaOH was added to the mixture until it reaches pH7.0). 75 mg in 133 mL volume equals to 0.056% (W/V) Carbopol 940. 1:1 ratio of TMB is added into each concentration before use. 4% (W/V) agar was added into water and autoclaved. Agar was then kept in 80° C. until needed. The appropriate amount of water and 1:1 ratio of TMB is added into each concentration before use.

ELISA plates were coated with Rabbit anti-human CD27 antibodies (0.5 μg/50 μL/well), made up in phosphate buffer saline (PBS) at pH7, minimum 12 hours before the experiment. The wells were then washed with PBS six times and blocked with RPMI 1640 containing 10% FCS.

On the day of the experiment, fresh blood samples were taken from volunteers. The blood was immediately diluted (50:50) in RPMI 1640 containing 1 mg/mL EDTA. Note: RPMI 1640 for cell culture work was conducted with RPMI 1640 with phenol red. Lymphocyte separation was conducted by taking the diluted blood and layered on top of 10 mL of lymphoprep, which was then centrifuged at 800 g for 30 mins with the brakes off. The lymphocyte layer was extracted using a sterile disposable pipette and fresh RPMI 1640+10% FCS+1 mg/mL EDTA+0.2% NaHCO₃ was added to dilute the lymphocyte containing lymphoprep. The lymphocyte was then centrifuged at 300 g for 10 mins. The supernatant was removed and fresh RPMI 1640+10% FCS+1 mg/mL EDTA+0.2% NaHCO₃ was added, which was then centrifuged at 300 g for 10 mins. This cleaning process was repeated 3 times.

Lymphocytes were then resuspended in RPMI 1640+10% FCS+1 mg/mL EDTA+0.2% NaHCO₃. 100 μL of the resuspended cells were taken and diluted in 900 μL RPMI 1640+10% FCS+1 mg/mL EDTA+0.2% NaHCO₃ (1 in 10 dilution). The cells were vortexed and 100 μL of this cell suspension was taken and added to 100 μL of trypan blue solution, which was then vortexed. 20 μL of trypan blue suspended cells were added onto a Neubauer improved counting chamber and cells were counted.

5×10⁵ cells/well were seeded in RPMI 1640+10% FCS+1 mg/mL EDTA+0.2% NaHCO₃ and incubated at 37° C. for 2 hours. After 2 hours, the wells were washed six times in warm (37° C.) PBS. Positive CD27 cells were further labelled with Rabbit anti-human CD27 antibodies (0.5 μg/50 μL/well) prepared in RPMI 1640+10% FCS+1 mg/mL EDTA+0.2% NaHCO₃. Control 1 wells were given RPMI 1640+10% FCS+1 mg/mL EDTA+0.2% NaHCO₃ only. The plates were then incubated at 37° C. for 2 hours. After 2 hours, the wells were washed six times in warm (37° C.) PBS.

The wells were treated with peroxidase suppressor for 15 mins at 4° C. followed by 10 mins at room temperature (RT). After peroxidase suppressor treatment, the wells were washed six times in warm (37° C.) PBS. HRP conjugated goat anti-rabbit Fc region antibodies, prepared 1 in 1000 dilution in RPMI 1640+10% FCS+1 mg/mL EDTA+0.2% NaHCO₃, were added to the sample wells. RPMI 1640+10% FCS+1 mg/mL EDTA+0.2% NaHCO₃ were added to the control 1 wells. The plates were left to incubate at RT for 1 hour. After 1 hour, the wells were washed six times in warm (37° C.) PBS.

A series of diffusion rate limiting mediums (DRLM) were prepared as well as PBS in place of the DRLM as Control 2; 0.3%, 1%, 2% (W/V) agar; 0.028% and 0.014% of Carbopol 940; 50% glycerol, all containing 50% TMB40 μL of each diffusion rate limiting medium (developing medium) were added to the appropriate wells and left to develop at 37° C. for 30 mins.

### Results

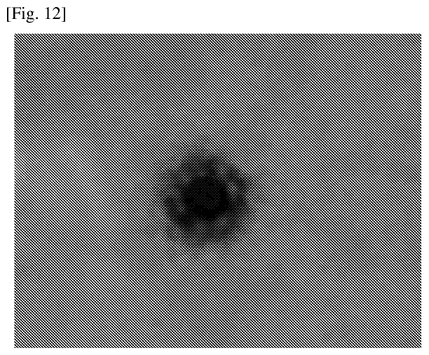

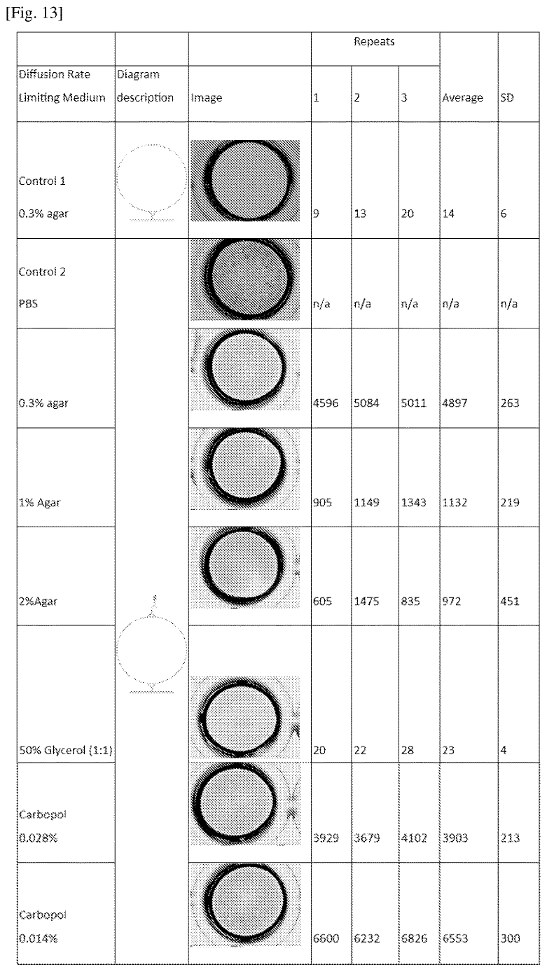

The different DRLMs tested are listed in [FIG. 13]. Very few background visible spots (dots) were seen in control 1 where there is no reporter enzyme probe added. Note between 9 and 20 spots were seen as background or false positives, likely due to endogenous peroxidase activity. In control 2 big clumps of TMB precipitation were observed with little to no clearly defined visible spots were formed and thus was not countable. In the case of agar and Carbopol, a wide range of visible spot (dot) counts can be observed between the different concentrations used. Average visible sport (dot) counts of 972±451, 1132±219 and 4897±263 for 2%, 1% and 0.3% W/V agar preparations, respectively were observed. Whereas average visible spot (dot) counts of 3903±213 and 6553±300 for 0.028% and 0.014% W/V Carbopol preparations were observed, respectively. 50% glycerol samples show very low number of visible spots (dots) ranging from 20 to 28 dots which is insignificantly different from control 1 due to the enzyme stabilising property of glycerol. (n=3).

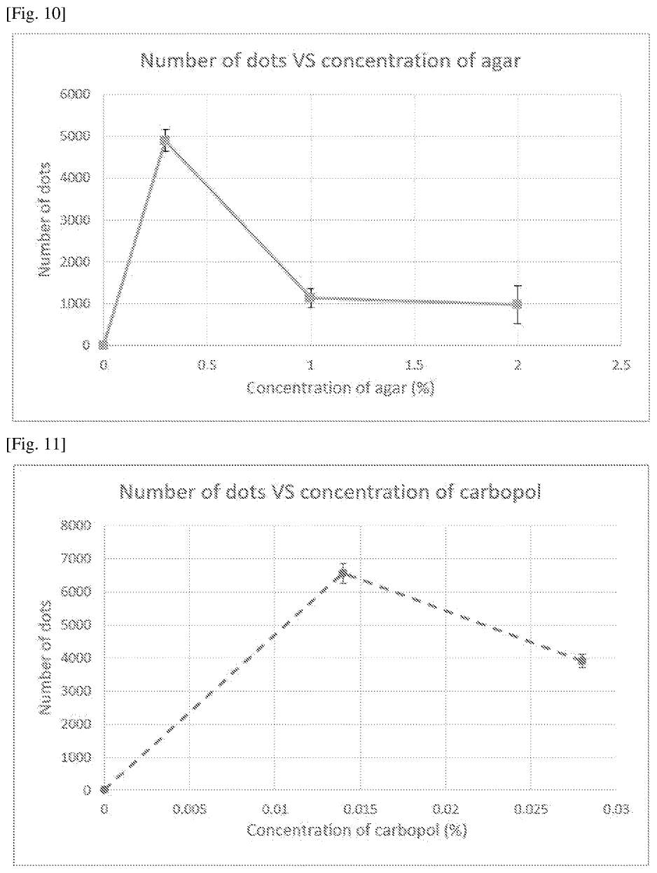

Control 1 which is not coupled with enzyme prob has an average of 14 visible spots (dots) caused by the endogenous peroxides which are known to be expressed in B cells [1]). The peroxide suppressor could not completely inhibit all endogenous peroxides activities of the cells however, the number of visible spots (dots) in control 1 is still significantly lower than in wells that incubated with the enzyme prob. Control 2 with PBS with 50% TMB has too much large TMB precipitation clumps to do any accurate counts. In addition, the whole solution turns blue/purple. This is because there was no DRLM in the substrate mixture to slow down the substrate/detectable product diffusion rate. The % (W/V) of agar and Carbopol greatly impact on the visible spot (dot) counts, with high % (W/V) agar (1-2% W/V) (see [FIG. 10]) and Carbopol (0.028% W/V) (see [FIG. 11]) content achieving lower visible spot (dot) counts and vice versa. The slower detectable product diffusion rate allows the concentration around the cell to build up to the point this is visible. However, if the diffusion rate is slowed down too much it inhibits the visible spot (dot) formation most likely due to unwanted substrate diffusion rate limiting thus insufficient substrate at the reaction site and product diffusion too slow to reach a visible size. It was shown that the higher concentration of the diffusion rate limiting medium gives a slower diffusion rate of methylene blue in above Example 1. It is important to note that the visible spot (dot) count for the 50% glycerol-TMB experiment is similar to that of the negative control, with very few visible and countable spots (dots). Glycerol is known to be an enzyme stabiliser causing glycerol-induced conformational changes responsible for the stability of the enzyme (in enzyme storage) 12) and inhibition (possibly due to hydrogen bond network disruption) (3). Glycerol serves as an example of a non-compatible media which is a viscous liquid that could slow down diffusion but incompatible for enzymatic reactions.

Microscopy study of the ELISA wells using an upward looking microscope, see [FIG. 12], at 100× magnification show the visible spot (dot) has a dark blue core with lighter blue/green halo irradiating from the core. This has shown that each dot is generated by one cell. Note: the visible spot (dot) is slightly bigger in size of a typical lymphocyte with one dot equalling one cell.

### Conclusion

Visible spots (dots) can be formed in the DRLM such as semi-solid (agar) or viscous liquid (Carbopol), as long as the medium is compatible with the experiment. A non-compatible medium, 50% glycerol-TMB, demonstrated visible spots (dots) cannot be formed due to incompatibility with the enzyme reaction. The % (W/V) of the semi-solid or the viscous liquid composition greatly impact on the visible spot (dot) count formation which is due to the diffusion of the chromogen substrate to the enzyme site and the detectable product from the enzyme site.

Using an ELISA plate platform allow for upward looking microscopy work (unlike ELISpot platform which as a solid membrane bottom). At 100× magnification, the visible spot (dot) appear to have a dark core with light blue/green halo representing the concentration of the blue chromogen product, where the light blue/green halo is the diffusion front of the product from the enzyme site. The dot is larger than a typical lymphocyte at ×100 magnification, with one dot representing one cell.

### REFERENCE

1. Okada, S. S. et al. (2016) ‘Myeloperoxidase in human peripheral blood lymphocytes: Production and subcellular localization’, Cellular Immunology, 300, pp. 18-25. doi: 10.1016/j.cellimm.2015.11.003.
2. Ramm, I. et al. (2021) ‘The Impact of Glycerol on an Affibody Conformation and Its Correlation to Chemical Degradation’, Pharmaceutics, 13 (11), pp. 1853-1866. https:/doi.org/10.3390/pharmaceutics13111853
3. Meneses, L. et al (2023) ‘Improving the activity of horseradish peroxidase in betaine-based natural deep eutectic systems’, RSC Sustainability, 1, pp. 886-897. https:/doi.org/10.1039/d2su00127f

### 4—Assay in Non-Immobilised Target Organisms (Cells)

### Aim:

To prove that the method of the invention also works on non-immobilised target organisms (cells).

### Materials

Rabbit anti-human CD27 antibodies, HRP conjugated goat anti-rabbit Fc region antibodies and Lymphoprep were purchased from Abcam. Phosphate buffer saline (PBS), Foetal Calf Serum (FCS), RPMI 1640 with phenol red, Ethylenediaminete traacetic acid (EDTA), sodium bicarbonate (NaHCO₃) and Peroxidase suppressor was purchased from ThermoFisher Scientific. Trypan blue solution, RPMI 1640 without phenol red and agar was purchased from Merck/Sigma-Aldrich. ELISA plates were purchased from Jet Biofil. 3,3′,5,5′-Tetramethylbenzidine (TMB) was purchased from Mabtech. Agarose was purchased from Bio Basic.

The macro ccd camera that was used to take the image was purchased from sh-renyue including calibration software S-eye. Data processing software, Icy software including Image J, was downloaded from https:/icy.bioimageanalysis.org/.

### Method

On the day of the experiment, fresh blood samples were taken from volunteers. The blood was immediately diluted (50:50) in RPMI 1640 containing 1 mg/ml EDTA. Note: RPMI 1640 for cell culture work was conducted with RPMI 1640 with phenol red. Lymphocyte separation was conducted by taking the diluted blood and layered on top of 10 mL of lymphoprep, which was then centrifuged at 800 g for 30 mins with the brakes off. The lymphocyte layer was extracted using a sterile disposable pipette and fresh RPMI 1640+10% FCS+1 mg/mL EDTA+0.2% NaHCO₃ was added to dilute the lymphocyte containing lymphoprep. The lymphocyte was centrifuged at 300 g for 10 mins. The supernatant was removed and fresh RPMI 1640+10% FCS+1 mg/mL EDTA+0.2% NaHCO₃ was added, which was then centrifuged at 300 g for 10 mins. This cleaning process was repeated 3 times.

Lymphocytes were then resuspended RPMI 1640+10% FCS+1 mg/mL EDTA+0.2% NaHCO₃. 100 μL of the resuspended cells were taken and diluted in 900 μL RPMI 1640+10% FCS+1 mg/mL EDTA+0.2% NaHCO₃ (1 in 10 dilution). The cells were vortexed and 100 μL of this cell suspension was taken and added to 100 μL of trypan blue solution, which was then vortexed. 20 μL of trypan blue suspended cells were added onto a Neubauer improved counting chamber and cells were counted.

Positive CD27 cells were labelled with Rabbit anti-human CD27 antibodies at 0.5 μg/5×10⁵ cell prepared in RPMI 1640+10% FCS+1 mg/mL EDTA+0.2% NaHCO₃ using a 50 mL falcon tube. Negative control cells were given RPMI 1640+10% FCS+1 mg/mL EDTA+0.2% NaHCO₃ only. The labelled and negative control cells were then incubated at 37° C. for 2 hours. After 2 hours, the cells were pelleted by centrifugation at 300 g for 10 mins, followed by washing with warm (37° C.) PBS and re-pelleted. This cell wash process was repeated 3 times.

The cells were treated with peroxidase suppressor for 15 mins at 4° C. followed by 10 mins at room temperature (RT). After peroxidase suppressor treatment, the cells were washed by centrifugation at 300 g for 10 mins, followed by wash with warm (37° C.) PBS and re-pelleted. This cell wash process was repeated 3 times.

HRP conjugated goat anti-rabbit Fc region antibodies, prepared 1 in 1000 dilution in RPMI 1640+10% FCS+1 mg/mL EDTA+0.2% NaHCO₃, were added to the Rabbit anti-human CD27 antibodies labelled cells and RPMI 1640+10% FCS+1 mg/mL EDTA+0.2% NaHCO₃ were added to the negative control wells. The cells were left to incubate at RT for 1 hour. After 1 hour, the cells were washed by centrifugation at 300 g for 10 mins, followed by wash with warm (37° C.) PBS and re-pelleted. This cell wash process was repeated 3 times.

0.028% (W/V) Carbopol-TMB was prepared by mixing 0.058% (W/V) Carbopol with TMB at 1:1 ratio. TMB solution specific for ELISA and ELISpot assays were both tested. The 0.028% (W/V) Carbopol-TMB mixture was added directly to the cell pellet, vortex and directly add to a microscope slide (10 μL). The microscope slides were left to develop at 37° C. for 30 mins. Photos were taken using a macro ccd camera.

### Result

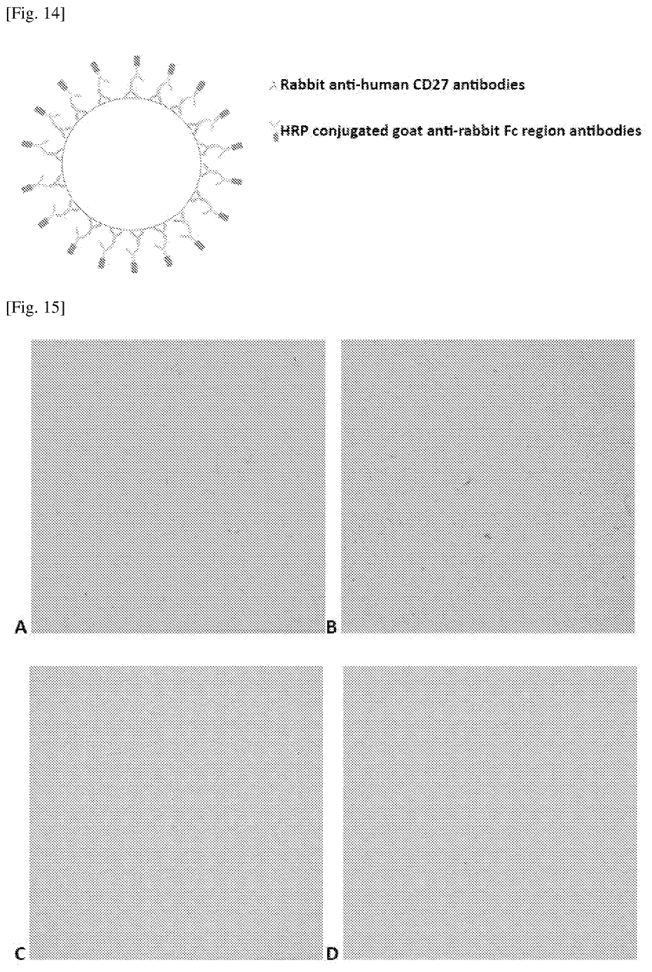

Non-immobilised CD27 labelled cells, as diagrammatically depicted in [FIG. 14], showing the cell fully labelled with primary Rabbit anti-human CD27 antibodies followed by secondary HRP conjugated goat anti-rabbit Fc region antibodies. When mixed with [FIG. 15] A) ELISA-specific TMB and B) ELISpot-specific TMB solutions both developed dots. The negative controls (non-immobilised unlabelled cells) [FIG. 15] C) ELISA-specific TMB and D) ELISpot-specific TMB solutions show very few visible spots (background).

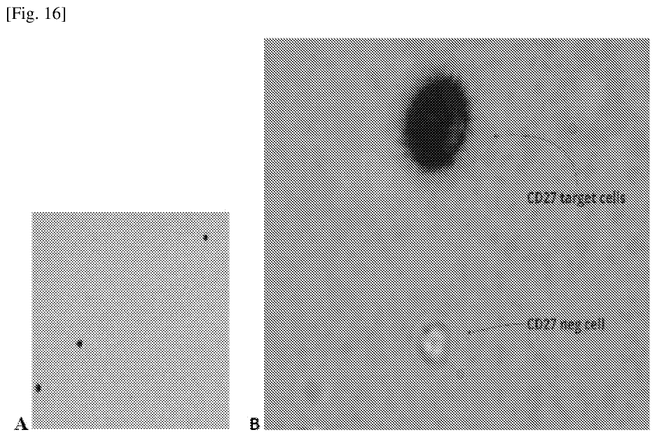

Microscopy study of the microscope slides, [FIG. 16] A), at ×10 magnification multiple visible spots (dots) are visible. More importantly, at ×100 magnification (FIG. 16B)) the visible spot (dot) has a dark blue core with a light blue/green halo surrounding the visible sport (dot) which is the diffusion front. In addition, when compared to a nearby non-target cell, the dot covers a volume that is bigger in size than a cell.

### Conclusion

The method of the invention provides visible spots (dots) with non-immobilised cells that have been labelled with primary Rabbit anti-human CD27 antibodies followed by HRP conjugated goat anti-rabbit Fc region antibodies. Each visible spot (dot) represents a labelled cell grown from enzymatic reaction of the chromogen to a visible chromophore, in this example horse radish peroxidase (HRP) is the enzyme and 3,3′,5,5′-Tetramethylbenzidine (TMB) substrate is the chromogen. The diffusion front of the chromophore (3,3′,5,5′-tetramethylbenzidine diamine) which is less concentrated compared to the reaction site (or the core) appears as a light blue/green halo. The core of the reaction site (surface label of the cell) appears almost black-to-dark blue in colour. The visible spot (dot) is bigger than the labelled cells due to the diffusion effect of the chromophore radiating outwards from the core of the reaction site. A direct comparison of a CD27 labelled dot versus a CD27 negative cell show the visible spot (dot) are much bigger in size in comparison to the CD27 negative cell. This demonstrates the diffusion rate of the diffusion rate limiting medium is essential in the formation of larger more easily detectible visible spots (dots) and the colour formed is not just due to cell staining.

### 5—Specific Antigen Targeting Memory B Cells Assay: SARS-COV-2 Spike Protein and Nucleocapsid Protein.

### Aim:

To detect antigen-specific memory B cells in peripheral blood mononuclear cells (PBMC) against SARS-Cov-2 spike protein and nucleocapsid protein as a model.

### Materials

Rabbit anti-human CD27 antibodies and Lymphoprep were purchased from Abcam. Phosphate buffer saline (PBS), Fetal Calf Serum (FCS), RPMI 1640 with phenol red, Ethylenediaminetetraacetic acid (EDTA), sodium bicarbonate (NaHCO₃) and Peroxidase suppressor was purchased from ThermoFisher Scientific. ELISpot plates, trypan blue solution, RPMI 1640 without phenol red and agar was purchased from Merck/Sigma-Aldrich. 3,3′,5,5′-Tetramethylbenzidine (TMB) was purchased from Mabtech. Biotinylated SARS-Cov-2 Spike protein (from the Wuhan-Hu-1 isolate) and biotinylated nucleocapsid protein (2019-nCov WHU02 isolate) were purchased from Acro Biosystems.

The macro ccd camera that was used to take the image was purchased from sh-renyue including calibration software S-eye. Data processing software, Icy software including Image J, was downloaded from https:/icy.bioimageanalysis.org/.

### Method

ELISpot plates were coated with Rabbit anti-human CD27 antibodies (0.5 μg/50 μL/well), made up in PBS at pH7, minimum 12 hours before the experiment. The wells were then washed with PBS six times and blocked with RPMI 1640 containing 10% FCS.

On the day of the experiment, fresh blood samples were taken from volunteers. The blood was immediately diluted (50:50) in RPMI 1640 containing 1 mg/ml EDTA. Note: RPMI 1640 for cell culture work was conducted with RPMI 1640 with phenol red. Lymphocyte separation was conducted by taking the diluted blood and layered on top of 10 mL of lymphoprep, which was then centrifuged at 800 g for 30 mins with the brakes off. The lymphocyte layer was extracted using a sterile disposable pipette and fresh RPMI 1640+10% FCS+1 mg/mL EDTA+0.2% NaHCO₃ was added to dilute the lymphocyte containing lymphoprep. The lymphocyte was centrifuged at 300 g for 10 mins. The supernatant was removed and fresh RPMI 1640+10% FCS+1 mg/mL EDTA+0.2% NaHCO₃ was added, which was then centrifuged at 300 g for 10 mins. This cleaning process was repeated 3 times.

Lymphocytes were then resuspended RPMI 1640+10% FCS+1 mg/mL EDTA+0.2% NaHCO₃. 100 μL of the resuspended cells were taken and diluted in 900 μL RPMI 1640+10% FCS+1 mg/mL EDTA+0.2% NaHCO₃ (1 in 10 dilution). The cells were vortexed and 100 μL of this cell suspension was taken and added to 100 μL of trypan blue solution, which was then vortexed. 20 μL of trypan blue suspended cells were added onto a Neubauer improved counting chamber and cells were counted.

5×10⁵ cells/well were seeded in RPMI 1640+10% FCS+1 mg/mL EDTA+0.2% NaHCO₃ and incubated at 37° C. for 2 hours. After 2 hours, the wells were washed six times in warm (37° C.) PBS. Positive CD27 cells were further labelled with biotinylated SARS-Cov-2 S protein and biotinylated nucleocapsid protein (100 ng/50 μL/well of each antigen) prepared in RPMI 1640+10% FCS+1 mg/mL EDTA+0.2% NaHCO₃. Negative control wells were given RPMI 1640+10% FCS+1 mg/mL EDTA+0.2% NaHCO₃ only. The plates were then incubated at 37° C. for 2 hours. After 2 hours, the wells were washed six times in warm (37° C.) PBS.

The wells were treated with peroxidase suppressor for 15 mins at 4° C. followed by 10 mins at room temperature (RT). After peroxidase suppressor treatment, the wells were washed six times in warm (37° C.) PBS. HRP conjugated streptavidin (1 mg/mL) stock was diluted 1 in 1000 in RPMI 1640+10% FCS+1 mg/mL EDTA+0.2% NaHCO₃ was added into test wells, and RPMI 1640+10% FCS+1 mg/mL EDTA+0.2% NaHCO₃ was added to the negative control wells. The plates were left to incubate at 37° C. for 45 mins. After 45 mins, the wells were washed six times in warm (37° C.) PBS.

The diffusion rate limiting medium (DRLM) composed of agar, ELISpot specific TMB and 4×RPMI without phenol red was prepared by taking 1:1:2 ratio of 4×RPMI: 1.2% Agar—warmed to 80° C.: stock TMB solution leading to a 0.3% agar/RPMI-TMB semi-solid medium. 40 μL of the semi-solid TMB medium was added to each well. The plates were then incubated at 37° C. for 30 mins. Photos were taken using a macro ccd camera.

### Result

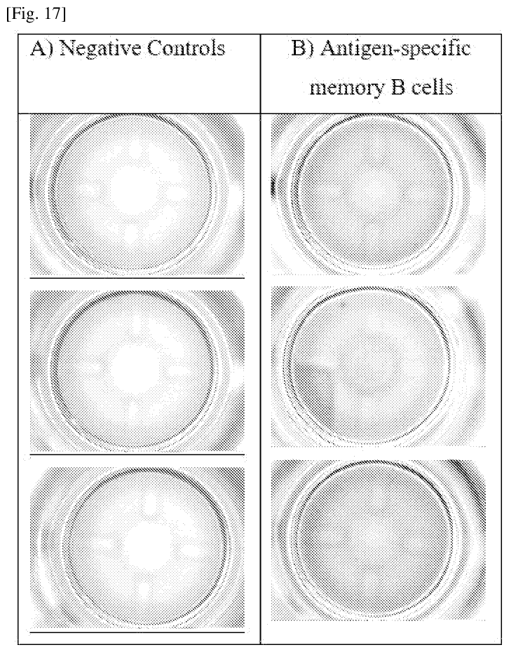

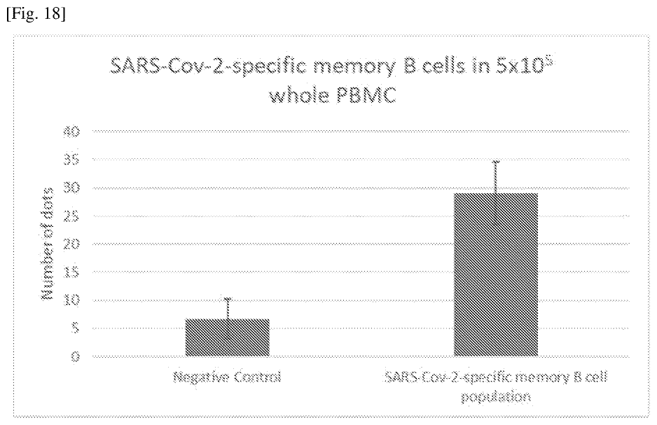

CD27 positive cells were immobilised onto the experimental wells with Rabbit anti-human CD27 antibodies. These immobilised CD27 positive cells from peripheral blood samples includes T cells^{[1] [3]}, natural killer cells^{[2] [3]}, memory B cells and plasma cells but not naïve B cells [4]. Negative control cells were not labelled with SARS-Cov-2 spike and nucleocapsid proteins (see FIG. 17A)). Whereas SARS-Cov-2-specific memory B cells are labelled with biotinylated SARS-Cov-2 spike and nucleocapsid proteins (see FIG. 17B)). Out of all the immobilised CD27+ cells only cells that expresses B cell antigen receptors (BCR), can directly bind to the targeted antigen without MHC molecule^{[5] [6]}. Plasma B cells lack of surface immunoglobulin expression^{[7]}, which left memory B cell the only cells that are immobilised on the plate surface expressing BCR and can be tagged by the antigen-enzyme prob and generate visible spots (dots). Visibly, there appear to be some background visible spots (dots) in the negative control tests, however there are significantly more visible spots (dots) in the test wells, and this is confirmed with visible spot (dot) counts shown in Table 2 and histogram plot of the data in [FIG. 18].

[Table 2] Summarises the visible spot (dot) counts for each experimental well shown in [FIG. 17], including the average and standard deviation between the triplicates, n=3.

| | | Repeats | | | |
|---|---|---|---|---|---|
| Experimental Description | 1 | 2 | 3 | Average | SD |
| Negative Control | 3 | 7 | 10 | 7 | 4 |
| SARS-Cov-2-specific | 34 | 30 | 23 | 29 | 6 |
| memory B cell population | | | | | |

### Conclusion

In this study, a direct approach for detecting antigen-specific memory B cells in peripheral blood using the method of the invention is provided. The method was able to determine the frequency of SARS-Cov-2 antigen specific memory B cells to SARS-Cov-2 spike and nucleocapsid proteins (average antigen-specific memory B cells dot count (29)—negative control average (7)=22/5×10⁵×100=0.0044%) from whole peripheral blood mononuclear cells (PBMC). This is in line with literature work of limiting dilution analyses of antibody-secreting cell precursor frequency is between 0.05-0.005% depending on vaccination status and the size/number of epitopes present on the antigen^{[8]}.

### REFERENCES

1. Hintzen, R Q et al. “Regulation of CD27 expression on subsets of mature T-lymphocytes.” Journal of immunology (Baltimore, Md.: 1950) vol. 151,5 (1993): 2426-35.
2. Silva, Anabel et al. “Application of CD27 as a marker for distinguishing human NK cell subsets.” International immunology vol. 20,4 (2008): 625-30. doi: 10.1093/intimm/dxn022.
3. Turaj A H, Hussain K, Cox K L, Rose-Zerilli M J J, Testa J, Dahal L N, Chan HTC, James S, Field V L, Carter M J, Kim H J, West J J, Thomas L J, He L Z, Keler T, Johnson P W M, Al-Shamkhani A, Thirdborough S M, Beers S A, Cragg M S, Glennie M J, Lim S H. Antibody Tumor Targeting Is Enhanced by CD27 Agonists through Myeloid Recruitment. Cancer Cell. 2017 Dec. 11; 32 (6): 777-791.e6. doi: 10.1016/j.ccell.2017. 11.001. Epub 2017 Nov. 30. PMID: 29198913; PMCID: PMC5734932.
4. Levesque, Marc C, and E William St Clair. “B cell-directed therapies for autoimmune disease and correlates of disease response and relapse.” The Journal of allergy and clinical immunology vol. 121,1 (2008): 13-21; quiz 22-3. doi: 10.1016/j.jaci.2007.11.030
5. Alberts B, Johnson A, Lewis J, et al. Molecular Biology of the Cell. 4th edition. New York: Garland Science; 2002. T Cells and MHC Proteins.
6. Paul S, Lal G. The Molecular Mechanism of Natural Killer Cells Function and Its Importance in Cancer Immunotherapy. Front Immunol. 2017 Sep. 13; 8:1124. doi: 10.3389/fimmu.2017.01124. PMID: 28955340; PMCID: PMC5601256.
7. Ribatti, D. (2017). The discovery of plasma cells: An historical note. Immunology Letters, 188, pp. 64-67. doi: https:/doi.org/10.1016/j.imlet.2017.06.006.
8. Smith M J, Packard T A, O'Neill S K, Hinman R M, Rihanek M, Gottlieb P A, Cambier J C. Detection and Enrichment of Rare Antigen-specific B Cells for Analysis of Phenotype and Function. J Vis Exp. 2017 Feb. 16; (120): 55382. doi: 10.3791/55382. PMID: 28287549; PMCID: PMC5409333.

1. A method for detecting a target organism in a sample, the method comprising
a. optionally immobilizing the target organism to a solid phase;
b. incubating the target organism with one or more reporter enzyme detection probes to form one or more target organism-enzyme detection probe complexes;
c. removing any unbound reporter enzyme detection probes;
d. contacting the one or more target organism-enzyme detection probe complexes with a diffusion rate limiting medium containing one or more substrates and incubating the one or more target organism—enzyme detection probe complexes with the one or more substrates in the diffusion rate limiting medium to generate one or more detectable products; and
e. detecting the one or more detectable products by detecting visible spots;
wherein the diffusion rate limiting medium is any suitable medium that decreases diffusion rate of the one or more detectable products and thereby allowing formation of the visible spots.

2. The method of claim 1, wherein step b) comprises incubating the target organism with one or more primary detection agents specific for the target organism prior to incubating with the one or more reporter enzyme detection probe to form the one or more target-enzyme detection probe complexes.

3. The method of claim 2, wherein the one or more primary detection agents bind to one or more surface ligands of the target organism, one or more surface markers of the target organism, or one or more target substances that are secreted by the target organism, and wherein the primary detection agent is an antibody or a binding fragment thereof, or an antigen.

4. The method of claim 1, wherein if the target organism expresses endogenous enzymes that are the same or similar to the reporter enzymes of the one or more reporter enzyme detection probes, the step b) further comprises contacting the target organism with suppressors of said endogenous enzymes.

5. The method of claim 1, wherein the target organism is a cell or a part thereof, or a microorganism or a part thereof.

6. The method of claim 1, wherein the solid phase is a reaction vessel, a bead or a plate, and wherein the surface of the solid phase is selected from the group consisting of metal, gold, stainless steel, plastic, glass, silica, polycarbonate, polyester, PVDF, polystyrene, nitrocellulose, and cellulose.

7. The method of claim 1, wherein the target organism is immobilized by directly binding the solid phase or is immobilized to the solid phase indirectly by a capture molecule coupled to the solid phase that binds the target organism.

8. The method of claim 7, wherein the capture molecule is an antibody, an antigen, or a ligand.

9. The method of claim 1, wherein the diffusion rate limiting medium is a semi-solid medium, a membrane or a viscous fluid.

10. The method of claim 9, wherein the semi-solid medium is selected from the group comprising consisting of agar, agarose, hydrogel, gelatine, silicone gel, cellulose derivatives, and SDS-PAGE gel.

11. The method of claim 9, wherein the membrane is selected from the group consisting of a paper membrane, a fiber membrane, a cellulose membrane and a plastic membrane.

12. The method of claim 9, wherein the viscous fluid is selected from the group consisting of a cellulose derivative, a polysaccharide or monosaccharide gel, and a low concentration hydrogel.

13. The method of claim 1, wherein the one or more reporter enzyme detection probes bind to one or more surface ligands of the target organism, one or more surface markers of the target organism, one or more target substances that are secreted by the target organism or the one or more primary detection agents, and wherein the reporter enzyme detection probe is selected from antibodies or binding fragment thereof, antigens, drugs, or peptides, which are directly or indirectly conjugated to an enzyme or a catalyst that can convert a color in contact with the substrate.

14. The method of claim 1, wherein the substrate is selected from the group consisting of tetramethylbenzidine (TMB), 5-bromo-4-chloro-3-indolyl-beta-D-galacto-pyranoside (X-Gal), 5-bromo-4-chloro-3-indolyl phosphate (BCIP), p-nitrophenol (PNPP), 3,3′-Diaminobenzidine (DAB), 4-(Trifluoromethyl) umbelliferyl phosphate, 4-Methylumbelliferyl β-D-galactopyranoside (Mu-Gal), adamantyl 1,2-dioxetane phosphate, o-nitrophenyl-β-D-galactopyranoside (ONPG), Nitro blue tetrazolium (NBT), 2,2′-Azinobis [3-ethylbenzothiazoline-6-sulfonic acid]-diammonium salt (ABTS), o-phenylenediamine dihydrochloride (OPD), Nitro blue tetrazolium chloride (NBT), N-Acetyl-Leu-Glu-His-Asp-7-amino-4-Trifluoromethylcoumarin, N-Acetyl-Leu-Glu-Thr-Asp-7-amino-4-Trifluoromethylcoumarin, and 3-oxo-3H-phenoxazin-7-yl-butanoic acid ester (Resorufin butyrate).

15. A method of quantifying the amount of a target organism in a sample, the method comprising
a. detecting the target organism according to the method of claim 1; and
b. quantifying the amount of the target organism in the sample based on the number of visible spots.

16. The method of claim 10, wherein the hydrogel is selected from the group consisting of Carbopol 940 and 2NapFF.

17. The method of claim 12, wherein the cellulose derivative is carboxymethylcellulose.

18. The method of claim 12, wherein the polysaccharide or monosaccharide gel is selected from the group consisting of sugar syrup, low concentration hydrogel, cassava gel, guar gum, xanthan gum, acacia gum, hydroxyethylcellulose and HPC (hydroxypropyl cellulose).
